(19) United States
(12) Patent Application Publication
(10) Pub. No.: US 2026/0306677 A1
(43) Pub. Date: Oct. 8, 2026

(54) METHOD FOR DETECTING A GD2 POSITIVE CANCER

(71) Applicant: PRINSES MÁXIMA CENTRUM VOOR KINDERONCOLOGIE B.V., 3584 CS Utrecht (NL)

(72) Inventors: Marcus Henricus Wilhelmus Andreas WIJNEN, Utrecht (NL); Alida Francina Willemien VAN DER STEEG, Utrecht (NL); Anne Caroline RIOS, Utrecht (NL); Alexander L. VAHRMEIJER, Utrecht (NL)

(21) Appl. No.: 19/483,912

(22) PCT Filed: May 10, 2024
(86) PCT No.: PCT/EP2024/062991

§ 371 Date: Nov. 12, 2025

(30) Foreign Application Priority Data
May 12, 2023 (GB) 2307103.8

(51) Int. Cl. A61K 49/00

(52) U.S. Cl. CPC A61K 49/0058; A61K 49/0021

(57) ABSTRACT

A GD2-specific fluorescent agent for use in a method of detecting a GD2 positive tumour in a human patient; wherein the GD2-specific fluorescent agent comprises a conjugate of a near-infrared fluorescent dye IRDye800CW and a ch14.18 antibody; and wherein the method comprises intravenously administering the GD2-specific fluorescent agent to the patient in a dose of from 1 to 20 mg/m² body surface area commencing between 1 day and 7 days prior to detecting of the GD2 positive tumour; and wherein detecting the GD2 positive tumour comprises exposing the GD2 positive tumour to NIR light and detecting the fluorescence emission from the GD2 positive tumour. The method is also useful in fluorescence-guided resection of a GD2 positive tumour.

### FIELD OF THE INVENTION

The present invention relates to detection and more particularly to imaging and fluorescence-guided surgery of solid tumours, particularly involving use of a near-infrared fluorescent dye-antibody conjugate as tracking agent.

### BACKGROUND TO THE INVENTION

Treatment of solid tumours typically involves surgical resection, unless tumour removal would compromise essential anatomic structures. For a variety of solid tumours, surgery is believed to be essential for treatment to be curative. As described in Privitera L, Paraboschi I, Dixit D, Arthurs O J and Giuliani Stefano. “Image-guided surgery and novel intraoperative devices for enhanced visualisation in general and paediatric surgery: a review” Innovative Surgical Sciences, vol. 6, no. 4, 2021, pp. 161-172, fluorescence guided surgery (FGS), augmented reality, and intra-operative imaging devices are rapidly pervading the field of surgical interventions, equipping the surgeon with powerful tools capable of enhancing the surgical visualisation of anatomical normal and pathological structures. They report that FGS has proved to be a feasible tool for visualising vessels, organ perfusion, and tumours during surgical procedures, with high contrast and sensitivity, and good spatial resolution of fine anatomical structures. These factors could improve the real-time and high-resolution delineation of vital structures and tumour margins during surgery. In addition to FGS, fluorescent tracers may be used to detect tumours for diagnostic purposes, particularly for diagnostic imaging, because tracer fluorescence may be detectable through layers of surrounding tissue. Indocyanine green (IGC) and fluorescein sodium have been used in children and adults with no reported side-effects. Immunofluorescence is an emerging experimental technique in which fluorophore-labelled antibodies are employed for specific and targeted visualization. Although clinical trials have been conducted in various cancers, some have been terminated, and as yet no fluorophore-labelled antibody has as yet been approved by regulators in US or Europe. In addition, no clinical trials have yet been attempted in the pediatric population, although fluorescence-guided surgery has been suggested for treatment of pediatric Ewing, osteo- and rhabdomyosarcomas (Rijs Z et al. Biomedicines. 2021; 9 (10): 1388. https://doi.org/10.3390/biomedicines9101388.

Neuroblastoma, after brain cancer, is the most frequent solid cancer in children under 5 years of age. Over 75% of patients are categorized as high-risk with a poor overall 5-year survival of less than 50%, despite intensive treatment with high-dose chemotherapy followed by surgery, autologous stem cell transplantation, radiotherapy and immunotherapy, as described in Tas, M. L. et al. Neuroblastoma between 1990 and 2014 in the Netherlands: increased incidence and improved survival of high-risk neuroblastoma. Eur. J. Cancer 124, 47-55. https://doi.org/10.1016/j.ejca.2019.09.025 (2020). Approximately 90% of cases occur between ages 0 to 6 years. The worldwide incidence in industrialized countries is around 2000 cases per year. Resection of high-risk NB is associated with a risk for serious surgical complications, since the tumor often encases major blood vessels leading to severe hemorrhage and/or unplanned organ damage, as described in Fischer, J. et al. Complete surgical resection improves outcome in INRG high-risk patients with localized neuroblastoma older than 18 months. BMC Cancer 17, 520. https://doi.org/10.1186/s1288 5-017-3493-0 (2017); and Rich, B. S. et al. Resectability and operative morbidity after chemotherapy in neuroblastoma patients with encasement of major visceral arteries. J. Pediatr. Surg. 46, 103-107. https://doi.org/10.1016/j.jpedsurg.2010.09.075 (2011).

Immunotherapy of neuroblastoma typically involves monoclonal antibodies which bind to the disialoganglioside GD2, a glycosphingolipid expressed primarily on the cell surface. GD2 expression in normal tissues is rare and primarily restricted to the central nervous system (CNS), peripheral nerves, and melanocytes. In cancerous cells, GD2 is expressed in neuroblastomas and most melanomas and to a variable degree in bone and soft-tissue sarcomas, small-cell lung cancer, renal cell carcinoma, and brain tumors (Navid et al., Curr Cancer Drug Targets 2010). GD2 is also expressed in Ewing sarcoma (Kailayangiri S et al., Br J Cancer. 2012; 106 (6): 1123-1133. doi:10.1038/bjc.2012.57), osteosarcoma (Roth M et al, Cancer 2014, 120, 548-554, doi:10.1002/cncr.28461), breast cancer (Orsi G et al. Oncotarget 2017; 8:31592-31600), desmoplastic small round cell tumor (Dobrenkov K et al. Pediatr Blood Cancer. 2016; 63:1780-1785) and retinoblastoma (Fleurence J et al. Journal of Immunology Research, Volume 2017, Article ID 5604891, https://doi.org/10.1155/2017/5604891).

Antibody 14G2a has been developed as chimeric (murine/human) forms known as ch14.18, in particular, dinutuximab beta (Qarziba®) and dinutuximab (Unituxin®). Dinutuximab beta is produced in Chinese Hamster Ovary (CHO) cells, and Unituxin is produced in SP2/0 murine hybridoma cells. Dinutuximab beta, also referred to as ch14.18/CHO or APN311 is licensed in the European Union (EU) subject to additional monitoring as Qarziba®, and treatment consists of 5 consecutive courses, each course comprising 35 days. Two modes of administration are approved: a continuous infusion over the first 10 days of each course (a total of 240 hours) at the daily dose of 10 mg/m²; or 5 daily infusions of 20 mg/m² administered over 8 hours, on the first 5 days of each course. Dinutuximab beta is indicated for the treatment of high-risk neuroblastoma in patients aged 12 months and above, who have previously received induction chemotherapy and achieved at least a partial response, followed by myeloablative therapy and stem cell transplantation, as well as patients with history of relapsed or refractory neuroblastoma, with or without residual disease. Dinutuximab beta is also authorised as a medicinal product in Australia and Israel. Various patents cover methods of using Dinutuximab beta, particularly U.S. Pat. No. 9,777,068 B2 which discloses a continuous intravenous infusion regimen which reduces the side-effect of pain; and U.S. Pat. No. 9,840,566 B2 and U.S. Pat. No. 10,294,305 B2 which disclose treatment regimens in which IL-2 is not administered in the same treatment cycle or overall treatment period. Unituxin® is authorised for treatment of high-risk neuroblastoma in the US, in combination with IL-12. A humanized version of ch14.18 known as hu14.18K322A is described in WO2005/070967 and has a point mutation in the Fc region in order to reduce complement-dependent cytotoxicity (CDC) but still maintain antibody-dependent cellular cytotoxicity (ADCC).

Given its relative specificity for GD2 positive cancer tissue, compared to healthy tissue, fluorescently labelled anti-GD2 antibodies have been suggested as tracers for detecting and imaging neuroblastomas. However, none has yet been clinically trialled. Experiments in mouse xenograft models have been performed, as described in Wellens, LM, Deken, MM, Sier, C F M, et al. (2020) Anti-GD2-IRDye800CW as a targeted probe for fluorescence-guided surgery in neuroblastoma. Sci Rep. 10, 17667. In this study, neuroblastoma tumour cells or cell lines were injected into mice, as a model to image tumours with anti-GD2-IRDye800CW, but resulting tumours do not reflect the location or properties of tumours in human subjects. A further report describes fluorescent graphene quantum dots (GQD) with or without conjugation to anti-GD2 antibody, and in vivo targeting of neuroblastomas and imaging in a similar mouse xenograft neuroblastoma model in You-Sheng Lin, Yun Chen, Ya-Hui Tsai, Sheng-Hong Tseng, Kuen-Song Lin. In vivo imaging of neuroblastomas using GD2-targeting graphene quantum dots, Journal of Pediatric Surgery, Volume 56, Issue 7, 2021, Pages 1227-1232, https://doi.org/10.1016/j.jpedsurg.2021.03.035. Here, the authors conclude that as their animal model is a subcutaneous xenograft model, the in vivo experimental results could not be directly applied to the spontaneously occurring tumours in humans. In addition, there is as yet no clear way of translating the dose suitable for use in a mouse to one that would be optimal in a human subject.

There remains a need to improve surgical procedures and outcomes in patients with neuroblastoma or other GD2-positive tumours.

The listing or discussion of an apparently prior-published document in this specification should not necessarily be taken as an acknowledgement that the document is part of the state of the art or is common general knowledge.

### SUMMARY OF THE INVENTION

A first aspect of the invention provides a GD2-specific fluorescent agent for use in a method of detecting a GD2 positive tumour in a human patient;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
wherein detecting the GD2 positive tumour comprises exposing the GD2 positive tumour to NIR light and detecting the fluorescence emission from the GD2 positive tumour.

A second aspect provides a GD2-specific fluorescent agent for use in a method of diagnosing an area of resectable GD2 positive tumour in a human patient;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
wherein diagnosing an area of resectable GD2 positive tumour comprises exposing the GD2 positive tumour to NIR light and detecting the fluorescence emission from the GD2 positive tumour.

Alternatively, a second aspect provides a GD2-specific fluorescent agent for use in a method of diagnosing an area of resectable GD2 positive tumour in a human patient;
wherein the GD2-specific fluorescent agent comprises a conjugate of a near-infrared fluorescent dye IRDye800CW and a ch14.18 antibody; and
wherein the GD2-specific fluorescent agent has been intravenously administered to the patient in a dose of from 1 to 20 mg/m² body surface area commencing between 1 day and 7 days prior to detecting of the GD2 positive tumour; and
wherein diagnosing an area of resectable GD2 positive tumour comprises exposing the GD2 positive tumour to NIR light and detecting the fluorescence emission from the GD2 positive tumour.

A third aspect provides a GD2-specific fluorescent agent for use in a method of fluorescence guided surgery to resect a GD2 positive tumour;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
detecting the GD2 positive tumour by exposing the GD2 positive tumour to NIR light and detecting the fluorescence emission from the GD2 positive tumour; and
surgical resection of the GD2 positive tumour detected in the patient.

A fourth aspect provides a method of imaging a GD2 positive tumour, the method comprising exposing the GD2 positive tumour to NIR light and detecting the fluorescence emission from the GD2 positive tumour,
wherein the patient has previously been administered a GD2-specific fluorescent agent in a dose of from 1 to 20 mg/m² body surface area commencing between 1 day and 7 days prior to imaging of the GD2 positive tumour; and wherein the GD2-specific fluorescent agent comprises a conjugate of a near-infrared fluorescent dye IRDye800CW and a ch14.18 antibody.

### DESCRIPTION OF THE FIGURES

FIG. 1: Workflow of rolling 6 strategy (extension of 3+3 design). Based on the efficacy of the dose for the imaging during surgery per patient, it will be decided to move to a next, higher dose.

FIG. 2: Intra-operative imaging results will be presented as (A) a bright field image, (B) a black-and-white fluorescent image, (C) a fluorescent overlay image, and (D) a fluorescent heat-map (D). Sample images are of a tumor-positive lymph node during abdominal surgery.

FIG. 3: Mapping process of the ex vivo specimen. During step 1 the fresh tissue specimen is imaged ex vivo to ensure images of all angles using the Quest camera. After formalin fixation, the specimen is mapped and sliced into breadloaves in step 2. Once the breadloaves are generated, fluorescence imaging using the Quest camera is performed of the breadloaves separately. In step 4 cassettes are made and imaged on a fluorescence scanner. The cassettes are paraffin embedded and cut into microscopy slides. In step 5, these slides are used for pathology assessment and fluorescence scanning.

FIG. 4: Direct correlation of fluorescence with histology and biomarkers of tumour cells is performed by evaluating sequential slides.

### DETAILED DESCRIPTION OF THE INVENTION

The present invention provides a method of detecting a GD2 positive tumour in a human patient and a GD2-specific fluorescent agent for use in the method. The GD2-specific fluorescent agent is or comprises a conjugate of a near-infrared (NIR) fluorescent dye IRDye800CW and a ch14.18 antibody.

A ch14.18 antibody consists of 2 light chains (220 amino acids) and 2 heavy chains (443 amino acids) and is of the IgG1 subclass. The monoclonal antibody incorporates human constant regions for the heavy chain IgG1 and the kappa light chain, along with the mouse variable regions targeted specifically against human GD2. The relative molecular mass of the intact antibody is approximately 150,000 daltons. The encoding nucleotide sequences and the amino acid sequences of a ch14.18 antibody are provided in U.S. Pat. No. 9,777,068 B2. In particular, the light-chain nucleotide sequence is provided as SEQ ID NO.1, the heavy-chain nucleotide sequence is provided as SEQ ID NO.2, the light-chain amino acid sequence as SEQ ID NO. 3, and the heavy-chain amino acid sequence as SEQ ID NO. 4. The first 60 nucleotides of SEQ ID NO. 1 or SEQ ID NO. 2 encode the signal peptide of the heavy or light chain respectively. The first 20 amino acids of SEQ ID NO. 3 or 4 are the signal peptide of the heavy or light chain respectively. The signal peptides are cleaved off during post-translational processing, and are not part of the final recombinant protein.

Ch14.18 antibody may be produced in Chinese Hamster Ovary (CHO) cells, and is designated ch14.18/CHO or dinutuximab beta (Qarziba®). Methods of manufacture and formulation of dinutuximab beta for clinical use are described in the European Public Assessment Report (EPAR) of the Committee for Medicinal Products for Human Use (CHMP) of the European Medicines Agency (EMA) for Dinutuximab beta Apeiron (EMA/263814/2017, 23 Mar. 2017). Ch14.18 antibody may be produced in SP2/0 murine hybridoma cells, and is designated ch14.18/SP2/0 or dinutuximab (Unituxin®). Suitable ch14.18 antibodies for use of the invention are dinutuximab beta or dinutuximab.

The method comprises intravenously administering the GD2-specific fluorescent agent to the patient in a dose of from 1 to 20 mg/m² body surface area commencing between 1 day and 7 days prior to detecting of the GD2 positive tumour. For example, the GD2-specific fluorescent agent may be administered in a dose of 1, 2, 3, 4, 5, 6, 7, 8, 9, 10, 11, 12, 13, 14, 15, 16, 17, 18, 19 or 20 mg/m² body surface area, typically at 1, 3 or 10 mg/m² body surface area. The units of dose are expressed in mg/m², where the area (in m²) refers to the patient's body surface area (BSA). For example, if a patient has a body surface area of 0.7 m², then a dose of 1 mg/m² would be 0.7 mg; a dose of 3 mg/m² would be 2.1 mg; and a dose of 10 mg/m² would be 7 mg.

The GD2-specific fluorescent agent may be provided as a composition, which may further comprise e.g. water for injection (WFI). The composition comprising the GD2-specific fluorescent agent may further comprise a buffer, e.g., histidine buffer or phosphate-buffered saline. The composition may further comprise stabilizing agents, preservatives and other carriers or excipients. The composition may comprise histidine, sucrose, polysorbate 20 and WFI. The composition may be stored as a solution in a refrigerator. The composition may be freeze-dried and reconstituted for use. The GD2-specific fluorescent agent should be prepared under sterile conditions. It is recommended that the GD2-specific fluorescent agent solution is filtered (0.2 to 1.2 μm) before injection into the patient either by using an in-line filter during infusion or by filtering the solution with a particle filter (e.g., filter Nr. MF1830, Impromediform, Germany). The volume of the GD2-specific fluorescent agent is typically diluted aseptically with an appropriate volume of NaCl 0.9% and 1% human serum albumin, for example in an infusion bag or syringe. A daily dose of 10 mg/m² is typically infused in a volume of 48 ml.

The GD2-specific fluorescent agent is typically administered by intravenous infusion, optionally over a period of 24 hours. Alternatively, it may be administered over fewer than 24 hours per day, such as over between 8 and 24 hours, or over 8 hours, or over 4 hours. The intravenous administration commences between 1 day and 7 days prior to detection of the GD2 positive tumour. Thus, if the administration is over a period of 24 hours, and commences two days prior to detection of the GD2 positive tumour, the detection is performed or commences one day after the intravenous administration of the GD2-specific fluorescent agent is completed. There is usually a gap of at least 16 h, at least 20 h, at least 24 hours, or at least 2 days between completion of the intravenous administration and detection of the GD2 positive tumour. Typically, the administering of the GD2-specific fluorescent agent is commenced 4 days prior to detecting the GD2 positive tumour. Thus, typically, the GD2-specific fluorescent agent may be administered in a dose of 1, 2, 3, 4, 5, 6, 7, 8, 9, 10, 11, 12, 13, 14, 15, 16, 17, 18, 19 or 20 mg/m² body surface area, more typically at 1, 3 or 10 mg/m² body surface area, commencing 4 days prior to detecting the GD2 positive tumour.

The method of detecting the GD2 positive tumour comprises exposing the GD2 positive tumour to NIR light and detecting the fluorescence emission from the GD2 positive tumour. By NIR light, we include light having a wavelength ranging from about 700 nm to about 2,500 nm, particularly from 700 to 900 nm, or from 780 to 900 nm. A NIR camera system is typically used for illumination and detection of fluorescence emission, and may acquire (NIR) fluorescence and white light images simultaneously allowing for a depicted overlay. A suitable NIR camera system for surgical use is the Artemis camera system described in van Driel P B, van de Giessen M, Boonstra M C, Snoeks T J, Keereweer S, Oliveira S, van de Velde C J, Lelieveldt B P, Vahrmeijer A L, Löwik CW, Dijkstra J. Characterization and evaluation of the artemis camera for fluorescence-guided cancer surgery. Mol Imaging Biol. 2015 June; 17 (3): 413-23. doi:10.1007/s11307-014-0799-z. PMID: 25344146; PMCID: PMC4422838. A laser is typically used to provide NIR light, such as with a peak intensity at 785 nm or 793 nm. A suitable filter is used to collect excitation light, such as a 800-850 nm band pass filter.

In methods of detection, imaging and fluorescence guided surgery, a tumor to background ratio (TBR) of the fluorescence signal of at least 2.0 has been found to be satisfactory in Wellens et al, supra. Thus, in the present methods, TBR of the fluorescence signal is typically at least 2.0, such as between 2.0 and 50, between 2.0 and 20, between 2.0 and 10, between 2.0 and 5.0, between 2.0 and 4.0, or between 2.0 and 3.0. The TBR may be least 2.5, at least 3.0, at least 4.0 or at least 5.0, and up to 50 or even 100. When the TBR is at least 2.0, the distinction between fluorescent and non-fluorescent tissue can be detected visually. The TBR is conveniently determined ex vivo in surgically removed tumour tissue, i.e. as an ex vivo TBR. TBR is established by dividing the mean fluorescence signal of the tumor by the mean fluorescence signal of the background, as described in the Example. Background is determined by fluorescence intensity in healthy kidney, liver and surrounding background tissue in the patient. The mean fluorescence signal of the resected tumour and the healthy tissue is typically determined using the same camera system. For a given conjugate, the mean fluorescence signal of the tumour depends on extent of conjugate accumulation in the tumour, which depends on a number of factors including the abundance of antigen to which the conjugate binds (in this case GD2), the interval between fluorescence measurement and administration of the conjugate, and the dose administered. The mean fluorescence signal of the background generally reflects the low rate of natural autofluorescence, and the non-specific presence of the conjugate in the absence of antigen to which it binds (i.e. GD2). The TBR for a standardised conjugate administration and detection/imaging scheme can be expected to be the same, whether it involves surgery or not. Thus, although it may be experimentally determined ex vivo, once the conditions required to achieve an ex vivo TBR of at least 2.0 are determined, these are applicable throughout surgical and non-surgical methods. In the event that the TBR might vary between different GD2 positive cancers, the criterion TBR of at least 2.0 is to be determined in high risk neuroblastoma.

As a diagnostic for GD2 positive vital tumour cells, the GD2 specific fluorescent agent as used in the methods is characterized by the following parameters, which have the following definitions:

| | |
|---|---|
| Sensitivity | Proportion of GD2 positive lesions which are |
| | also fluorescent |
| Positive predictive | Proportion fluorescent lesions that contain GD2 |
| value | expressing tumor cells |
| Specificity | Proportion of non-malignant tissue that is not |
| | fluorescent (and GD2 negative) |

These diagnostic parameters are evaluated by determining pathologic status of removed fluorescent tissue and removed non-fluorescent tissue and comparing to fluorescent status. As described in the Example, ex vivo resected specimen fluorescence hotspots and removed non-fluorescent tissue will be assessed for fluorescence intensity; and pathologic analysis based on morphological characteristics will differentiate between different neuroblastoma stages, vital tumor cells and/or necrosis. The pathology morphology report of these samples will be compared with the mean fluorescence intensity. Samples will be processed as described in the Example. Upon excision during surgery, the entire fresh specimen will be cut into slices and imaged by the optical imaging system in overlay images of white light and fluorescence mode, in order to identify fluorescence hotspots. Next, tissue will be formalin fixed, mapped and sliced into breadloaves, fluorescence scanned, then embedded into paraffin blocks and fluorescence scanned. Overlay images of white light and fluorescence will be taken, to track the fluorescence. Paraffin blocks will be processed into thin sections e.g. 5 μm on slides for histologic evaluation by H&E staining and immunohistochemistry for GD2 expression using standard techniques, and will be assessed by an experienced pediatric oncology pathologist according to the INPC classification. This will be compared to fluorescence intensity of slides, or alternatively of the paraffin blocks or breadloaves from which they are derived. The immunohistochemistry provides an independent assessment of the ‘condition’ to be diagnosed i.e. GD2 positive vital tumour lesion, which is compared to the ‘test result’ of fluorescence. The ‘condition’ is scored as present or absent for any given location on a slide, and the ‘test result’ is scored as positive or negative. Given that certain healthy tissues may express GD2, these are excluded from the determination of specificity, and can be ignored by clinicians in carrying out the detection methods. The histologic and fluorescence analysis is typically performed on sequential slides to rigorously correlate the test result with the condition. The correlation may then be reasoned backwards to the paraffin block and breadloaf from which the slides were prepared. Thus, it is also possible to evaluate the test result of fluorescence in the paraffin block or breadloaf, with the histologic evaluation performed on the slides. Alternatively, the immunohistochemistry, histology and fluorescence intensity may be evaluated using fresh tissue sections obtained from the breadloaves. A mixed strategy may also be possible, in which sections of the breadloaves are processed by embedding in paraffin blocks, and other sections are processed as fresh tissue cryosections.

The diagnostic parameters are then calculated from the number of true positives, false positives, false negatives and true negatives by constructing a 2×2 contingency table as illustrated below, and reproduced from Bruno P. The importance of diagnostic test parameters in the interpretation of clinical test findings: The Prone Hip Extension Test as an example. J Can Chiropr Assoc. 2011 June; 55 (2): 69-75. PMID: 21629460; PMCID: PMC3095582:

Condition

| | | Present | Absent |
|---|---|---|---|
| Test Result | Positive | a | b |
| | Negative | c | d |

a = “True Positives”
b = “False Positives”
c = “False Negatives”
d = “True Negatives”
Sensitivity = a/(a + c)
Specificity = d/(b + d)
Positive predictive value = a/(a + b)
Negative predictive value = d/(c + d)

The sensitivity of a diagnostic test ranges in principle from 0 to 1. A high sensitivity indicates a high proportion of true positives and a small proportion of false negatives. The specificity of a diagnostic test ranges in principle from 0 to 1. A high specificity indicates a high proportion of true negatives and a small proportion of false positives. The positive predictive value (PPV) of a diagnostic test ranges in principle from 0 to 1. A high PPV indicates a high proportion of true positives and a small proportion of false positives. These parameters may alternatively be expressed as percentages.

Diagnostic parameters may be averaged over different patients under like conditions i.e. who have received the same dose of GD2-specific fluorescent agent, at the same time interval prior to tumour detection. The diagnostic parameters for a standardised conjugate administration and detection/imaging scheme can be expected to be the same, whether it involves surgery or not. Thus, although they may be experimentally determined ex vivo, once the diagnostic parameters are determined, these can be expected to be applicable throughout surgical and non-surgical methods. In the event that the diagnostic parameters might vary between different GD2 positive cancers, the parameters are to be determined in high risk neuroblastoma and the values indicated are to be understood accordingly.

Typically, the sensitivity of the GD2-specific fluorescent agent for the detection of GD2-expressing tumor cells, typically GD2-expressing vital tumor cells, is at least 0.1, at least or up to 0.2, at least or up to 0.3, at least or up to 0.4, at least or up to 0.5, at least or up to 0.6, at least or up to 0.65, at least or up to 0.7, at least or up to 0.75, at least or up to 0.8, at least or up to 0.85, at least or up to 0.9, or at least or up to 0.95. Sensitivity may fall within a range defined by any of the above limits.

Typically, the specificity of the GD2-specific fluorescent agent for the detection of GD2-expressing tumor cells, typically GD2-expressing vital tumor cells, is at least 0.5, at least 0.6, at least 0.65, at least 0.7, at least 0.75, at least 0.8, at least 0.85, at least 0.9, at least 0.95, at least 0.96, or at least 0.97, at least 0.98, or at least 0.99. A specificity of at least 0.95 is preferred and typical of diagnostic tests.

Typically, the positive predictive value of the GD2-specific fluorescent agent for the detection of GD2-expressing tumor cells, typically GD2-expressing vital tumor cells, is at least 0.5, at least or up to 0.6, at least or up to 0.65, at least or up to 0.7, at least or up to 0.75, at least or up to 0.8, at least or up to 0.85, at least or up to 0.9, at least or up to 0.95, at least or up to 0.96, at least or up to 0.97, at least or up to 0.98, or at least or up to 0.99. PPV may fall within a range defined by any of the above limits.

Typically, the conjugate is formed by reacting an activated IRDye800CW dye moiety with a ch14.18 antibody. A suitable activated dye moiety is a IRDye800CW ester, such as a N-hydroxysuccinimide (NHS) ester. Typically, the ch14.18 antibody is conjugated to the IRDye800CW by an amide bond. This is typically formed between a carboxylic acid group on the dye moiety and a free amine on an amino acid residue of the antibody. One or more than one dye moiety may become conjugated to the antibody, depending on the conjugation conditions. The degree of labelling (DOL) specifies how many dye molecules are attached to one antibody molecule. A DOL may be less than, greater than or equal to 1.0. A DOL may be between 3.0 and 1.0, typically between 1.5 and 1.0. A DOL may be between 0.8 and 1.2, such as between 0.9 and 1.1. A DOL may be about 0.85, about 0.90, about 0.95, about 1.0, about 1.05 or about 1.10. Suitable conjugation conditions and methods for calculating dye/protein ratio and protein concentration are described in IRDye® 800CW Protein Labeling Kit—High MW, Li-Cor, Inc., Lincoln, Nebraska 68504 USA, June 2012. A suitable conjugation method is as used in Wellens et al, 2020, supra.

The GD2-positive cancer to be detected according to method is typically neuroblastoma. Alternatively to neuroblastoma, the patient may have another GD2-positive cancer, such as melanoma, a bone or soft-tissue sarcoma, small-cell lung cancer, renal cell carcinoma, a brain tumor, Ewing sarcoma, osteosarcoma, breast cancer, desmoplastic small round cell tumor, or retinoblastoma. The presence of GD2 expression may be determined using immunohistochemistry, for example on biopsy specimens. The patient may be a pediatric patient (age<18 years) or and adult patient.

Typically, the patient has neuroblastoma, such as high risk neuroblastoma, although other risk classifications are also contemplated. The patient may have newly-diagnosed, relapsed, resistant or refractory neuroblastoma.

Neuroblastomas are cancers that start in early nerve cells (called neuroblasts) of the sympathetic nervous system, and they can be found anywhere along this system. Most primary tumors (65%) occur within the abdomen with at least half of these arising in the adrenal medulla. Other common sites of disease include the neck, chest, and pelvis. Presenting signs and symptoms are highly variable and dependent on site of primary tumor as well as the presence or absence of metastatic disease and/or paraneoplastic syndromes. International Neuroblastoma Risk Group Staging System (INRGSS) is a clinical classification system that is determined prior to any treatment, including surgery, based on preoperative imaging. It classifies according to 2 stages of localized (L1 and L2) and 2 stages of metastatic disease (M and MS) (Monclair T et al (2009) J. Clin. Oncol., 27:298-303, 2009). A new International Neuroblastoma Risk Group (INRG) classification system has been proposed in 2009 with 4 broad categories-very low risk, low risk, intermediate risk, and high risk-based on the assessment of the following prognostic factors: age at diagnosis (2 cutoffs, 12 and 18 months), INRG tumour stage (L1, L2, M, MS), histologic category, grade of tumour differentiation, DNA ploidy (hyperploidy/diploidy), MYCN oncogene status (amplified or not), aberrations at chromosome 11q (presence/absence) (Cohn S L et al, Journal of Clinical Oncology 2009 27:2, 289-297). The INRG staging system evolved based on presurgical radiology and metastatic status. In this system, nonmetastatic tumors are assessed for surgical risk factors that predict unresectability using radiographic imaging, known as image-defined risk factors (IDRFs). This system uses combinations of the seven prognostic risk factors to define 16 pretreatment groups stratified by the prognostic markers within four categories, namely very low-, low-, intermediate- and or high-risk group, the categories based on the 5-year event-free survival (EFS) rates of the 16 pretreatment groups. The pretreatment groups (labeled A to R) and risk categories are summarized in Pinto N R et al, J Clin Oncol. 2015 Sep. 20; 33 (27): 3008-3017, as shown in Table 1.

TABLE 1
Neuroblastoma pretreatment groups and risk categories

| INRG | Age | Histologic | Grade of Tumor | | 11q | | Pre-treatment |
|---|---|---|---|---|---|---|---|
| Stage | (months) | Category | Differentiation | MYCN | Aberration | Ploidy | Risk Group |
| L1/L2 | | GN maturing, | | | | | A (very low) |
| | | GNB | | | | | |
| | | intermixed | | | | | |
| L1 | | Any, except | | NA | | | B (very low) |
| | | GN maturing | | Amplified | | | K (high) |
| | | or GNB | | | | | |
| | | intermixed | | | | | |
| L2 | <18 | Any, except | | NA | No | | D (low) |
| | | GN maturing | | | Yes | | G |
| | | or GNB | | | | | (intermediate) |
| | | intermixed | | | | | |
| | ≥18 | GNB nodular | Differentiating | NA | No | | E (low) |
| | | neuroblastoma | | | Yes | | H (intermediate) |
| | | | Poorly | NA | | | H |
| | | | differentiated or | | | | (intermediate) |
| | | | undifferentiated | Amplified | | | N (high) |
| M | <18 | | | NA | | Hyperdiploid | F (low) |
| | <12 | | | NA | | Diploid | I (intermediate) |
| | 12 to <18 | | | NA | | Diploid | J (intermediate) |
| | <18 | | | Amplified | | | O (high) |
| | ≥18 | | | | | | P (high) |
| MS | <18 | | | NA | No | | C (very low) |
| | | | | | Yes | | Q (high) |
| | | | | Amplified | | | R (high) |

By newly diagnosed neuroblastoma, we mean a first diagnosis of neuroblastoma within a patient, for which the patient has yet to receive any neuroblastoma treatment. The newly diagnosed neuroblastoma may fall within any of the INRGSS disease classifications, or it may have been diagnosed by any other clinically accepted means. Typically, a newly diagnosed neuroblastoma patient will commence treatment within a few weeks from diagnosis, but we do not intend the term “newly diagnosed” to imply any limit on the interval between diagnosis and treatment. Neuroblastoma may alternatively be detected in relapsed, refractory or resistant patients, according to the method. By “refractory”, or “treatment-refractory” disease, we mean signs or symptoms of disease that never improved or responded to treatment and simply progressed. By “resistant” or “treatment-resistant” disease, we mean signs or symptoms of disease that improved on or responded to treatment then returned. “Resistant” disease includes disease for which there has been at least a partial or minor response to prior treatment. By “relapsed” disease, we include disease that has returned following a complete response to surgical or nonsurgical treatment. The terms “resistant” and “relapsed” may be used interchangeably in some terminologies, as encompassing disease that has progressed or returned following a partial, minor, or complete response to prior therapy.

Detection of neuroblastoma according to the method may suitably be used in patients for whom surgical intervention may be contemplated, e.g. high-risk neuroblastoma, according to the high-risk classification of the International Neuroblastoma Risk Group (INRG) classification system, as described in Pinto N R et al, supra. In particular, pretreatment groups K, N, O, P, Q and R are identified as belonging to the high-risk classification, and the methods may be used to detect neuroblastoma in patients in any of these groups, which may be defined as any patient with MYCN amplified neuroblastoma (other than stage L1 according to INRGSS) or any patient older than 12 months of age at diagnosis with stage M disease. Suitable patients may be pretreatment group P (i.e. stage M, according to INRGSS, age≥18 months), and typically <18 years.

Suitably, detecting the GD positive tumour, typically neuroblastoma, comprises imaging the GD2 positive tumour. This may be carried out using a suitable camera system as described herein.

Suitably, the method comprises fluorescence guided surgery of the GD2 positive tumour. Surgery is typically performed in treatment of low, intermediate and high risk neuroblastoma, as set out in Maris J M. N Engl J Med. 2010; 362 (23): 2202-2211. Doi: 10.1056/NEJMra0804577) as follows:

TABLE 1
Phenotypic and Genetic Features of Neuroblastoma, Treatment,
and Survival According to Prognostic Category

| | Prognostic Category* | | | |
|---|---|---|---|---|
| Variable | Low Risk | Intermediate Risk | High Risk | Tumor Stage 4S |
| Pattern of | Localized | Localized tumor | Metastases to | Metastases to |
| disease | tumor | with | bone marrow and | liver and skin |
| | | locoregional | bone (except in | (with minimal |
| | | lymph-node | infants) | bone marrow |
| | | extension; | | involvement) |
| | | metastases to | | in infants |
| | | bone marrow | | |
| | | and bone in | | |
| | | infants | | |
| Tumor | Whole- | Whole- | Segmental | Whole- |
| genomics | chromosome | chromosome | chromosomal | chromosome |
| | gains | gains | aberrations | gains |
| Treatment | Surgery^{†} | Moderate- | Dose-intensive | Supportive |
| | | intensity | chemotherapy, | care^{‡} |
| | | chemotherapy; | surgery, and | |
| | | surgery^{†} | external-beam | |
| | | | radiotherapy to | |
| | | | primary tumor | |
| | | | and resistant | |
| | | | metastatic sites; | |
| | | | myeloablative | |
| | | | chemotherapy | |
| | | | with autologous | |
| | | | hematopoietic | |
| | | | stem-cell rescue; | |
| | | | isotretinoin with | |
| | | | anti-GD2 | |
| | | | immunotherapy | |
| Survival | >98 | 90 to 95 | 40 to 50 | >90 |
| rate (%) | | | | |

*Patients are categorized into prognostic groups according to risk, as described by the Children's Oncology Group, with the level of risk defining the likelihood of death from disease. Stage 4S disease is considered separately here because of the unique phenotype of favorable biologic features and relentless early progression but ultimately full and complete regression of disease.
^{†}The goal of surgery is to safely debulk the tumor mass and avoid damage to surrounding normal structures while also obtaining sufficient material for molecular diagnostic studies. Some localized tumors may spontaneously regress without surgery.
^{‡}Low-dose chemotherapy, radiation therapy, or both are used in patients with life- threatening hepatic involvement, especially in infants under 2 months of age, who are at much higher risk for life-threatening complications from massive hepatomegaly.

Detection, imaging or fluorescence-guided surgery may be used in any of these neuroblastoma risk categories, such as in newly diagnosed patients. Use in high risk neuroblastoma, such as in newly diagnosed patients is preferred given the low survival rate. Patients may have localized or metastatic disease.

Surgery may typically be performed in conjunction with induction chemotherapy. Chemotherapy for neuroblastoma typically involves multiple cycles of treatments with combinations of drugs. By induction therapy, we mean the first (frontline) or only course of chemotherapy administered to a newly diagnosed neuroblastoma patient. Induction chemotherapy may be administered before or after surgery, or surgery may be performed between treatment periods with induction chemotherapy drugs. Surgery may be performed more than once in conjunction with induction chemotherapy. Depending on risk classification, treatment may stop after induction chemotherapy and surgery. Induction therapy is typically used in the treatment of high-risk neuroblastoma, although patients classified as intermediate-risk may also receive chemotherapy and surgery (Pinto N R et al, 2015, supra).

Multimodal standard treatment for newly diagnosed high risk neuroblastoma may involve induction chemotherapy, megatherapy, radiation, surgery, and consolidation therapy. For high-risk neuroblastomas, the current treatment can be divided into 3 distinct phases (Maris J M. N Engl J Med. 2010; 362 (23): 2202-2211. Doi: 10.1056/NEJMra0804577): (i) induction of remission with intensive chemotherapy. After a response to chemotherapy, resection of the primary tumour is usually attempted. (ii) consolidation of the remission with myeloablative chemotherapy which attempts to eradicate minimal residual disease using lethal doses of chemotherapy followed rapidly by rescue with autologous hematopoietic progenitor cells to repopulate the bone marrow. (iii) a maintenance phase used to treat potential minimal residual disease (MRD) following HSCT to reduce the risk of relapse (Matthay K K et al. [published correction appears in J Clin Oncol. 2014 Jun. 10; 32 (17): 1862-3]. J Clin Oncol. 2009; 27 (7): 1007-1013. Doi: 10.1200/JCO.2007.13.8925), e.g., with dinutuximab beta and isotretinoin, a molecule that induces terminal differentiation of neuroblastoma cell lines.

In general, during induction, patients receive 5-8 cycles of chemotherapy including platinum compounds, alkylating agents, and topoisomerase inhibitors (Smith, V., & Foster, J. (2018). High-Risk Neuroblastoma Treatment Review. Children (Basel, Switzerland), 5 (9), 114. https://doi.org/10.3390/children5090114). The rapid COJEC regimen is the preferred European induction regimen from the SIOPEN group, described in Ladenstein R et al. J Clin Oncol. 2010; 28 (21): 3516-3524, and comprises consecutive chemotherapy cycles of A (vincristine, carboplatin and etoposide), B (vincristine and cisplatin), C (vincristine, cyclophosphamide and etoposide), B, A, B, C, and B. The preferred German regimen since 1997 is GPOH (Gesellschaft für Pädiatrische Onkologie und Hämatologie) NB2004 N5/N6, which involves 6 alternating chemotherapy cycles of N5 (cisplatin, etoposide, and vindesine) and N6 (ifosfamide, vincristine, dacarbazine, and doxorubicin) (Simon T et al. Klin Padiatr. 2017; 229 (3): 147-67.6; and Berthold F, Lancet Oncol. 2005; 6 (9): 649-58). The preferred US induction regimen from the Children's Oncology Group (COG) (ANBL0532 program) is described in Park J R et al, J Clin Oncol 2011; 29 (33): 4351-57. ANBL/COG involves 2 cycles of topotecan and cyclophosphamide (400 mg/m²/d) for 5 days followed by 4 cycles of multiagent chemotherapy (Memorial Sloan-Kettering Cancer Center [MSKCC] regimen comprising alternating cisplatin/etoposide and cyclophosphamide plus doxorubicin/vincristine).

Neuroblastoma surgery and complications are discussed in Luo Y B, Cui X C, Yang L, Zhang D, Wang J X. Advances in the Surgical Treatment of Neuroblastoma. Chin Med J (Engl). 2018 Oct. 5; 131 (19): 2332-2337. Doi: 10.4103/0366-6999.241803. PMID: 30246719; PMCID: PMC6166470. Surgery typically involves resection of the primary tumour and/or debulking at metastatic sites. Surgical resection aims to achieve macroscopic tumor resection with minimal residual disease. If features of the tumor (site, size, and relationship with surrounding structures) indicate that surgical resection is not feasible without risk, presurgical chemotherapy may be administered to shrink the tumor and enable safer tumor resection. SIOPEN has identified several imaging features (IDRFs) potentially associated with surgery-related complications. IDRFs include tumor extension into a second body compartment, encasement of any large blood vessels, tracheal or large bronchial compression, involvement of major nerve roots (such as the brachial plexus), invasion of the spinal canal, or infiltration of the nearby kidneys, mesentery, pericardium, liver, diaphragm, or pancreas. Minimally invasive surgery (MIS) may be applied for resection of primary tumours without IDRFs. Otherwise, open surgery may be performed.

The method may comprise surgical resection of all surgically resectable GD2 positive tumours detected in the patient. These may be primary tumour and/or metastases. Surgical resection may be classified by extent as follows: complete resection (95-100%), gross total resection (90-95%), incomplete resection (50-90%), and biopsy (<50%). One or more than one surgical resection may be performed. If a gross total resection is performed, this may be followed by a further gross total resection, or a complete resection. If an incomplete resection is performed, this may be followed by a further incomplete resection, a gross total resection, or a complete resection. If a biopsy is performed, this may be followed by an incomplete resection, a gross total resection, or a complete resection.

The method may further comprise estimation of extent of tumor resection. Postoperative staging to assess the presence and extent of possible residual tumors may be surgeon-estimated, or may be carried out by computed tomography, magnetic resonance imaging or ultrasound, typically at between one and three months postoperatively, as described in BMC Cancer 17, 520. https://doi.org/10.1186/s1288 5-017-3493-0 (2017).

Subsequent treatment may be selected based on the extent of resection achieved. If the resection extent is at least 90%, the patient is not treated with boost radiation therapy, Whereas if the resection extent is less than 90%, the patient is treated with boost radiation therapy. Radiation therapy (radiotherapy) is typically administered before and/or after surgery, typically at a total dose of 21 or 21.6 Gy. Where a resection extent of less than 90% is achieved, residual tumour is typically detectable by MRI or CT scanning. Otherwise, with a resection extent of at least 90%, residual tumour is generally not detectable by such means. In cases of residual tumour detection, boost radiotherapy may delivered to the residual tumour in a total dose of 14 or 14.6 Gy (for a total dose of 36 Gy to the tumour site when combined with the prior radiotherapy), as described in Kevin X. Liu et al Journal of Clinical Oncology 2020 38:24, 2741-2752. Radiation dose is typically applied in two or more fractions once or twice per day until the total dose has been administered. Selection of suitable radiation doses is described in Jazmati D, Brualla L, Littooij A S, Webber B, Dieckmann K, Janssens G O, Simon T, Gaze M N, Merta J, Serrano A, Dietzsch S, Kramer P H, Wulff J, Boterberg T, Timmermann B. Overcoming inter-observer planning variability in target volume contouring and dose planning for high-risk neuroblastoma—a European multicenter effort of the SIOPEN radiotherapy committee. Radiother Oncol. 2023 April; 181:109464. Doi: 10.1016/j.radonc.2023.109464. Epub 2023 Jan. 11. PMID: 36640946.

Selection of patients to undergo immunotherapy with a ch14.18 antibody may be based in part on The sensitivity of the GD2-specific fluorescent agent for the detection of GD2-expressing tumor cells, typically GD2-expressing vital tumor cells. For example, if the sensitivity for a given diagnostic or prognostic classification of patient, or a given patient, is at least 0.3, at least 0.4, at least 0.5, at least 0.6, at least 0.7, at least 0.8, at least 0.9 or at least 0.95 the patient may be administered immunotherapy. For example, patients may undergo immunotherapy with one or more cycles of Dinutuximab beta as described for example in U.S. Pat. No. 9,777,068 B2, U.S. Pat. No. 9,840,566 B2 and U.S. Pat. No. 10,294,305 B2. Dinutuximab beta may be administered as a continuous infusion over the first 10 days of each cycle (a total of 240 hours) at the daily dose of 10 mg/m²; or 5 daily infusions of 20 mg/m² administered over 8 hours, on the first 5 days of each cycle. Typically, treatment consists of 2, 3, 4 or 5 consecutive cycles. Each cycle may comprise 35 days. Alternatively, patients may undergo immunotherapy with one or more cycles of Dinutuximab, for example as described in The United States Food and Drug Administration initial U.S. Approval for Unituxin™, 2015. Dinutuximab may be administered at 17.5 mg/m²/day as an intravenous infusion over 10 to 20 hours for 4 consecutive days for up to 5 cycles. Cycles may further comprise administration of granulocyte-macrophage colony-stimulating factor (GM-CSF) or interleukin-2 (IL-2). Conversely, if the sensitivity of the GD2-specific fluorescent agent for the detection of GD2-expressing tumor cells for a given diagnostic or prognostic classification of patient, or a given patient, is up to 0.5, up to 0.4, up to 0.3, up to 0.2 or between 0.2 and 0.1, then the patient may not be administered immunotherapy.

The fluorescence guided surgery may increase the proportion of patients for whom a resection extent of at least 90% can be achieved, compared to the proportion of patients having the same prognostic classification and receiving standard surgical resection, within the same overall treatment plan. For example, in a cohort of neuroblastoma patients, such as newly diagnosed high risk neuroblastoma patients, a greater proportion of patients receiving fluorescence guided surgery may achieve at least 90% tumour resection compared to those receiving standard surgical resection. The only difference in treatment is in the fluorescence guided surgery vs standard surgery, as the patients are otherwise treated according to the same overall treatment plan e.g. induction chemotherapy, radiotherapy etc. The proportion of patients achieving at least 90% tumour resection may be at least 5%, at least 10%, at least 15%, at least 20% or at least 25% more, for example. Thus, if 50% of patients receiving standard surgery achieve at least 90% tumour resection, the percentage of patients achieving at least 90% tumour resection with fluorescence guided surgery would be at least 52.5%, at least 55%, at least 57.5%, at least 60% or at least 62.5%, for example.

The fluorescence guided surgery may improve one or more clinical parameters of a cohort of patients compared to a cohort of patients having the same prognostic classification and receiving standard surgical resection, within the same overall treatment plan, optionally wherein the one of more clinical parameters are selected from overall response rate, complete response rate, partial response rate, primary tumour volume reduction, Curie score, event free survival and overall survival. This may be assessed in a cohort of neuroblastoma patients, such as newly diagnosed high risk neuroblastoma patients. International neuroblastoma response criteria are described in Park J R et al J Clin Oncol. 2017; 35 (22): 2580. Suitable clinical parameters are selected from overall response rate (ORR), complete response (CR) rate, partial response (PR) rate, primary tumour volume reduction, Curie score, event-free survival (EFS), and overall survival (OS), overall response during and after induction (primary tumor, metastases), and metastatic CR and PR rates. EFS and OS may be determined at 3 or 5 years. ORR includes CR, and PR.

Complete Response (CR) may be further defined as follows:
Complete disappearance of all measurable and evaluable disease,
no new lesions,
no disease-related symptoms, and/or
no evidence of evaluable disease, including, e.g., normalization of markers and/or other abnormal laboratory values.

In some embodiments, all measurable, evaluable, and non-evaluable lesions and sites must be assessed using the same technique as baseline.

Partial Response (PR) may be further defined as follows:
Applies only to patients with at least 1 measurable lesion.
Greater than or equal to 50% decrease under baseline in the sum of products of perpendicular diameters of all measurable lesions.
No progression of evaluable disease.
No new lesions.

The proportion of patients achieving any one of these clinical parameters may be at least 5%, at least 10%, at least 15%, at least 20% or at least 25% more, for example. Thus, if 50% of patients receiving standard surgery achieve a given clinical parameter, the percentage of patients achieving the same clinical parameter with fluorescence guided surgery would be at least 52.5%, at least 55%, at least 57.5%, at least 60% or at least 62.5%, for example.

The fluorescence guided surgery may reduce the risk of surgical complications of a cohort of patients compared to a cohort of patients having the same prognostic classification and receiving standard surgical resection. Surgical complications are common in neuroblastoma and may be serious, including fatal, as described in Luo Y B et al, 2018, supra. The proportion of patients experiencing a surgical complication may be at least 5%, at least 10%, at least 15%, at least 20% or at least 25% lower, for example. Thus, if 50% of patients receiving standard surgery experience a surgical complication, the percentage of patients experiencing a surgical complication with fluorescence guided surgery would be at most 47.5%, at most 45%, at most 42.5%, at most 40% or at most 37.5%, for example.

Preferences and options for a given aspect, feature or parameter of the invention should, unless the context dictates otherwise, be regarded as having been disclosed in combination with any and all preferences and options for all other aspects, features and parameters of the invention.

All documents are incorporated by reference in their entirety.

Embodiments of the invention will now be described in the following numbered paragraphs.
1. A method of detecting a GD2 positive tumour in a human patient;
the method comprising intravenously administering a GD2-specific fluorescent agent to a patient in a dose of from 1 to 20 mg/m² body surface area commencing between 1 day and 7 days prior to detecting of the GD2 positive tumour;
   wherein detecting the GD2 positive tumour comprises exposing the GD2 positive tumour to near-infrared (NIR) light and detecting the fluorescence emission from the GD2 positive tumour; and
wherein the GD2-specific fluorescent agent comprises a conjugate of a near-infrared fluorescent dye IRDye800CW and a ch14.18 antibody.
2 The method of embodiment 1, wherein the GD2-specific fluorescent agent is administered in a dose of 1, 3, 10, 15 or 20 mg/m² body surface area.
3. The method of embodiment 1 or 2, wherein the GD2-specific fluorescent agent is administered by intravenous infusion, optionally over a period of 24 hours.
4. The method of any preceding embodiment, wherein administering the GD2-specific fluorescent agent is commenced 4 days prior to detecting the GD2 positive tumour.
5. The method of any preceding embodiment, wherein the ex vivo tumor to background ratio (TBR) of the fluorescence signal is at least 2.0.
6. The method of any preceding embodiment, wherein the sensitivity of the GD2-specific fluorescent agent for the detection of GD2-expressing tumor cells, typically GD2-expressing vital tumor cells, is at least 0.1, at least 0.2, at least 0.3, at least 0.4, at least 0.5, at least 0.6, at least 0.65, at least 0.7, at least 0.75, at least 0.8, at least 0.85, at least 0.9, or at least 0.95.
7. The method of any preceding embodiment, wherein the specificity of the GD2-specific fluorescent agent for the detection of GD2-expressing tumor cells, typically GD2-expressing vital tumor cells, is at least 0.5, at least 0.6, at least 0.65, at least 0.7, at least 0.75, at least 0.8, at least 0.85, at least 0.9, at least 0.95, at least 0.96, or at least 0.97, at least 0.98, or at least 0.99.
8. The method of any preceding embodiment, wherein the positive predictive value of the GD2-specific fluorescent agent for the detection of GD2-expressing tumor cells, typically GD2-expressing vital tumor cells, is at least 0.5, at least 0.6, at least 0.65, at least 0.7, at least 0.75, at least 0.8, at least 0.85, at least 0.9, at least 0.95, at least 0.96, or at least 0.97, at least 0.98, or at least 0.99.
9. The method of any preceding embodiment, wherein the ch14.18 antibody is dinutuximab beta.
10. The method of any one of embodiments 1 to 8, wherein the ch14.18 antibody is dinutuximab.
11. The method of any one of the preceding embodiments, wherein the ch14.18 antibody is conjugated to the IRDye800CW by an amide bond.
12. The method of any preceding embodiment, wherein the patient has neuroblastoma, such as high risk neuroblastoma, intermediate risk neuroblastoma or low risk neuroblastoma.
13. The method of embodiment 12, wherein the patient has newly-diagnosed, relapsed or refractory neuroblastoma.
14. The method of any preceding embodiment, wherein detecting the neuroblastoma comprises imaging the GD2 positive tumour.
15. The method of embodiment 14, wherein the method comprises fluorescence guided surgery of the GD2 positive tumour.
16. The method of embodiment 15, wherein fluorescence guided surgery comprises surgical resection of all surgically resectable GD2 positive tumours detected in the patient.
17. The method of embodiment 16, wherein the method further comprises estimation of extent of tumor resection.
18. The method of embodiment 17, wherein if the resection extent is at least 90%, the patient is not treated with boost radiation therapy, whereas if the resection extent is less than 90%, the patient is treated with boost radiation therapy.
19. The method of any of embodiments 15 to 18, wherein the fluorescence guided surgery increases the proportion of patients for whom a resection extent of at least 90% can be achieved, compared to the proportion of patients having the same prognostic classification and receiving standard surgical resection, within the same overall treatment plan.
20. The method of any one of embodiments 15 to 19, wherein the fluorescence guided surgery improves one or more clinical parameters of a cohort of patients compared to a cohort of patients having the same prognostic classification and receiving standard surgical resection, within the same overall treatment plan, optionally wherein the one of more clinical parameters are selected from overall response rate, complete response rate, partial response rate, primary tumour volume reduction, Curie score, event free survival and overall survival.
21. The method of any of embodiments 15 to 20, wherein the fluorescence guided surgery reduces the risk of surgical complications of a cohort of patients compared to a cohort of patients having the same prognostic classification and receiving standard surgical resection.

### Sequences

```
<211> 723
<212> DNA
<213> Artificial
<220>
<223> artificial antibody chain
<400> 1
atggaagccc cagcgcagct tctcttcctc ctgctactct ggctcccaga taccactgga 60
gaaatagtga tgacgcagtc tccagccacc ctgtctgtgt ctccagggga aagagccacc 120
ctctcctgca gatctagtca gagtcttgta caccgtaatg gaaacaccta tttacattgg 180
tacctgcaga agccaggcca gtctccaaag ctcctgattc acaaagtttc caaccgattt 240
tctggggtcc cagacaggtt cagtggcagt ggatcaggga cagatttcac actcaagatc 300
agcagagtgg aggctgagga tctgggagtt tatttctgtt ctcaaagtac acatgttcct 360
ccgctcacgt tcggtgctgg gaccaagctg gagctgaaac gaactgtggc tgcaccatct 420
gtcttcatct tcccgccatc tgatgagcag ttgaaatctg gaactgcctc tgttgtgtgc 480
ctgctgaata acttctatcc cagagaggcc aaagtacagt ggaaggtgga taacgccctc 540
caatcgggta actcccagga gagtgtcaca gagcaggaca gcaaggacag cacctacagc 600
ctcagcagca ccctgacgct gagcaaagca gactacgaga aacacaaagt ctacgcctgc 660
gaagtcaccc atcagggcct gagctcgccc gtcacaaaga gcttcaacag gggagagtgt 720
tag 723
<210> 2
<211> 1389
<212> DNA
<213> Artificial
<220>
<223> artificial antibody chain
<400> 2
atgggatgga cctggatctt tattttaatc ctgtcggtaa ctacaggtgt ccactctgag 60
gtccaactgc tgcagtctgg acctgagctg gagaagcctg gcgcttcagt gatgatatcc 120
tgcaaggctt ctggttcctc attcactggc tacaacatga actgggtgag gcagaacatt 180
ggaaagagcc ttgaatggat tggagctatt gatccttact atggtggaac tagctacaac 240
cagaagttca agggcagggc cacattgact gtagacaaat cgtccagcac agcctacatg 300
cacctcaaga gcctgacatc tgaggactct gcagtctatt actgtgtaag cggaatggag 360
tactggggtc aaggaacctc agtcaccgtc tcctcagcct ccaccaaggg cccatcggtc 420
ttccccctgg caccctcctc caagagcacc tctgggggca cagcggccct gggctgcctg 480
gtcaaggact acttccccga accggtgacg gtgtcgtgga actcaggcgc cctgaccagc 540
ggcgtgcaca ccttcccggc tgtcctacag tcctcaggac tctactccct cagcagcgtg 600
gtgaccgtgc cctccagcag cttgggcacc cagacctaca tctgcaacgt gaatcacaag 660
cccagcaaca ccaaggtgga caagagagtt gagcccaaat cttgtgacaa aactcacaca 720
tgcccaccgt gcccagcacc tgaactcctg gggggaccgt cagtcttcct cttcccccca 780
aaacccaagg acaccctcat gatctcccgg acccctgagg tcacatgcgt ggtggtggac 840
gtgagccacg aagaccctga ggtcaagttc aactggtacg tggacggcgt ggaggtgcat 900
aatgccaaga caaagccgcg ggaggagcag tacaacagca cgtaccgtgt ggtcagcgtc 960
ctcaccgtcc tgcaccagga ctggctgaat ggcaaggagt acaagtgcaa ggtctccaac 1020
aaagccctcc cagcccccat cgagaaaacc atctccaaag ccaaagggca gccccgagaa 1080
ccacaggtgt acaccctgcc cccatcccgg gaggagatga ccaagaacca ggtcagcctg 1140
acctgcctgg tcaaaggctt ctatcccagc gacatcgccg tggagtggga gagcaatggg 1200
cagccggaga acaactacaa gaccacgcct cccgtgctgg actccgacgg ctccttcttc 1260
ctctatagca agctcaccgt ggacaagagc aggtggcagc aggggaacgt cttctcatgc 1320
tccgtgatgc atgaggctct gcacaaccac tacacgcaga agagcctctc cctgtccccg 1380
ggtaaatga 1389
<210> 3
<211> 240
<212> PRT
<213> Artificial
<220>
<223> artificial antibody chain
<400> 3
Met Glu Ala Pro Ala Gln Leu Leu Phe Leu Leu Leu Leu Trp Leu Pro
1 5 10 15
Asp Thr Thr Gly Glu Ile Val Met Thr Gln Ser Pro Ala Thr Leu Ser
                        20 25 30
Val Ser Pro Gly Glu Arg Ala Thr Leu Ser Cys Arg Ser Ser Gln Ser
                35 40 45
Leu Val His Arg Asn Gly Asn Thr Tyr Leu His Trp Tyr Leu Gln Lys
        50 55 60
Pro Gly Gln Ser Pro Lys Leu Leu Ile His Lys Val Ser Asn Arg Phe
65 70 75 80
Ser Gly Val Pro Asp Arg Phe Ser Gly Ser Gly Ser Gly Thr Asp Phe
                                85 90 95
Thr Leu Lys Ile Ser Arg Val Glu Ala Glu Asp Leu Gly Val Tyr Phe
                        100 105 110
Cys Ser Gln Ser Thr His Val Pro Pro Leu Thr Phe Gly Ala Gly Thr
                115 120 125
Lys Leu Glu Leu Lys Arg Thr Val Ala Ala Pro Ser Val Phe Ile Phe
        130 135 140
Pro Pro Ser Asp Glu Gln Leu Lys Ser Gly Thr Ala Ser Val Val Cys
145 150 155 160
Leu Leu Asn Asn Phe Tyr Pro Arg Glu Ala Lys Val Gln Trp Lys Val
                                165 170 175
Asp Asn Ala Leu Gln Ser Gly Asn Ser Gln Glu Ser Val Thr Glu Gln
                        180 185 190
Asp Ser Lys Asp Ser Thr Tyr Ser Leu Ser Ser Thr Leu Thr Leu Ser
                195 200 205
Lys Ala Asp Tyr Glu Lys His Lys Val Tyr Ala Cys Glu Val Thr His
        210 215 220
Gln Gly Leu Ser Ser Pro Val Thr Lys Ser Phe Asn Arg Gly Glu Cys
225 230 235 240
<210> 4 <211> 462
<212> PRT
<213> Artificial
<220>
<223> artificial antibody chain
<400> 4
Met Gly Trp Thr Trp Ile Phe Ile Leu Ile Leu Ser Val Thr Thr Gly
1 5 10 15
Val His Ser Glu Val Gln Leu Leu Gln Ser Gly Pro Glu Leu Glu Lys
                        20 25 30
Pro Gly Ala Ser Val Met Ile Ser Cys Lys Ala Ser Gly Ser Ser Phe
                35 40 45
Thr Gly Tyr Asn Met Asn Trp Val Arg Gln Asn Ile Gly Lys Ser Leu
        50 55 60
Glu Trp Ile Gly Ala Ile Asp Pro Tyr Tyr Gly Gly Thr Ser Tyr Asn
65 70 75 80
Gln Lys Phe Lys Gly Arg Ala Thr Leu Thr Val Asp Lys Ser Ser Ser
                                85 90 95
Thr Ala Tyr Met His Leu Lys Ser Leu Thr Ser Glu Asp Ser Ala Val
                        100 105 110
Tyr Tyr Cys Val Ser Gly Met Glu Tyr Trp Gly Gln Gly Thr Ser Val
                115 120 125
Thr Val Ser Ser Ala Ser Thr Lys Gly Pro Ser Val Phe Pro Leu Ala
        130 135 140
Pro Ser Ser Lys Ser Thr Ser Gly Gly Thr Ala Ala Leu Gly Cys Leu
145 150 155 160
Val Lys Asp Tyr Phe Pro Glu Pro Val Thr Val Ser Trp Asn Ser Gly
                                165 170 175
Ala Leu Thr Ser Gly Val His Thr Phe Pro Ala Val Leu Gln Ser Ser
                        180 185 190
Gly Leu Tyr Ser Leu Ser Ser Val Val Thr Val Pro Ser Ser Ser Leu
                195 200 205
Gly Thr Gln Thr Tyr Ile Cys Asn Val Asn His Lys Pro Ser Asn Thr
        210 215 220
Lys Val Asp Lys Arg Val Glu Pro Lys Ser Cys Asp Lys Thr His Thr
225 230 235 240
Cys Pro Pro Cys Pro Ala Pro Glu Leu Leu Gly Gly Pro Ser Val Phe
                                245 250 255
Leu Phe Pro Pro Lys Pro Lys Asp Thr Leu Met Ile Ser Arg Thr Pro
                        260 265 270
Glu Val Thr Cys Val Val Val Asp Val Ser His Glu Asp Pro Glu Val
                275 280 285
Lys Phe Asn Trp Tyr Val Asp Gly Val Glu Val His Asn Ala Lys Thr
        290 295 300
Lys Pro Arg Glu Glu Gln Tyr Asn Ser Thr Tyr Arg Val Val Ser Val
305 310 315 320
Leu Thr Val Leu His Gln Asp Trp Leu Asn Gly Lys Glu Tyr Lys Cys
                                325 330 335
Lys Val Ser Asn Lys Ala Leu Pro Ala Pro Ile Glu Lys Thr Ile Ser
                        340 345 350
Lys Ala Lys Gly Gln Pro Arg Glu Pro Gln Val Tyr Thr Leu Pro Pro
                355 360 365
Ser Arg Glu Glu Met Thr Lys Asn Gln Val Ser Leu Thr Cys Leu Val
        370 375 380
Lys Gly Phe Tyr Pro Ser Asp Ile Ala Val Glu Trp Glu Ser Asn Gly
385 390 395 400
Gln Pro Glu Asn Asn Tyr Lys Thr Thr Pro Pro Val Leu Asp Ser Asp
                                405 410 415
Gly Ser Phe Phe Leu Tyr Ser Lys Leu Thr Val Asp Lys Ser Arg Trp
                        420 425 430
Gln Gln Gly Asn Val Phe Ser Cys Ser Val Met His Glu Ala Leu His
                435 440 445
Asn His Tyr Thr Gln Lys Ser Leu Ser Leu Ser Pro Gly Lys
        450 455 460
```

The present invention will be further illustrated in the following examples, without any limitation thereto.

### EXAMPLES

### Example 1: A Phase I/II First-In-Human Imaging Study of Anti-GD2 IRDye800CW in Patients with Neuroblastoma

### 1. Objectives

### 1.1 Primary Objectives

To establish the safety of the anti-GD-2-IRDye800CW imaging agent in pediatric patients with neuroblastoma
To determine the efficacy and optimal dose of the imaging agent for the intraoperative detection of neuroblastoma using NIR fluorescence

### 1.2 Secondary Objectives

To assess the pharmacodynamic and pharmacokinetic characteristics of an i.v. injection of the conjugate anti-GD2-IRDye800CW
To correlate clinical imaging data with histopathology data to determine the accuracy for depicting GD-2 expressing vital tumor cells

### 1.3 Trial Endpoints

### Primary Endpoints:

Treatment related new adverse events after injection of the conjugate anti-GD2-IRDye800CW not to be expected from dinutuximab-alone infusion.
Tumor to background ratio (TBR) of at least 2.0 ex vivo and sufficient for detection during surgery. TBR is established by dividing the mean fluorescence signal of the tumor by the mean fluorescence signal of the background. Background is determined by fluorescence intensity in healthy kidney, liver and surrounding background tissue.

### Secondary Endpoints:

Changes in serum pharmacokinetics; blood samples will be taken at t=0 (just before infusion), t=24 h (after infusion), t=72 h (after infusion), t=96 h (immediately before surgery), t=120 h (24 h after surgery) and t=144 h (48 h after surgery).
The sensitivity and specificity of our novel fluorescent agent to detect GD2-expressing, vital tumor cells. Pathologic status of removed fluorescent tissue and of removed non-fluorescent tissue will be compared to fluorescent status.
   After surgery, the ex-vivo resected specimen fluorescence hotspots will be marked within the same day. Pathologic analysis based on morphological characteristics, between 1-10 days after surgery, will differentiate between different neuroblastoma stages, vital tumor cells and/or necrosis. The pathology morphology report of these samples will be compared with the mean fluorescence intensity.

### 2. Background

### 2.1 Study Disease

### Neuroblastoma Surgery is a Challenge for the Surgeon

Neuroblastoma is the most commonly diagnosed extracranial solid tumor in young children. Patients diagnosed with high risk neuroblastoma-approximately 50% of all neuroblastoma patients-currently have a survival chance of only 50% despite intensive therapy. Children with high risk neuroblastoma are generally treated with induction chemotherapy, followed by surgery, high dose chemotherapy with stem cell rescue, additional radiotherapy and immunotherapy with dinutuximab (anti-GD2 antibodies) (1). Tumor resection remains technically challenging because of its encasement of blood vessels and nerves, and the involvement of lymph-nodes. Ex vivo, the International Neuroblastoma Pathology Classification (INPC) describes four neuroblastoma subtypes, including neuroblastoma (NB), ganglioneuroblastoma-nodular (GNB-N) subtype, ganglioneuroblastoma-intermixed (GNB-I), and ganglioneuroma. The first is considered most malignant, while ganglioneuroma's are less malignant associated with better outcomes. (2) Most neuroblastomas contain these subtypes heterogeneously distributed. For the surgeon, it is impossible during surgery to differentiate between different subtypes. Furthermore, the percentages of complications (e.g. bleeding or unplanned nephrectomy) during neuroblastoma surgery is high (40%) (3).

The presence of residual tumor cells after resection is considered a strong predictor of tumor recurrence. Many studies in adult cancers, including breast, lung, prostate, bladder and colorectal cancers, show that positive margins, defined as the identification of tumor cells at the margin of a surgical specimen, are associated with local recurrence and indicate a poor prognosis. For children with neuroblastoma, the objective benefit of surgery is difficult to evaluate. A recent review on the role of surgery in high-stage neuroblastoma performed by the Children's Oncology Group (COG) shows a beneficial effect of radical surgery in decreasing the cumulative incidence of local relapse (4). It has been established that a surgeon-assessed resection extent≥90% of tumor significantly improves the event free survival of patients with neuroblastoma (5).

To assist the surgeon in decision making during neuroblastoma debulking surgery, to reduce the amount of surgical complications and to make sure the undifferentiated neuroblastoma cells can be visualized and resected during surgery, new intra-operative visualization tools need to be developed.

### Fluorescence Imaging

Recently, novel intra-operative imaging modalities have been developed to assist surgeons to identify cancerous tissue and delineate tumor margins, which helps to reduce damage to important structures such as nerves, blood vessels and increase the extend of tumor surgery. The most promising methodology is Fluorescence-guided surgery (FGS), which offers the benefit of high contrast and sensitivity and the absence of ionizing radiation (e.g. intraoperative computed tomography and/or radioactive colloids). In addition, it is easy to use and does not disrupt the normal workflow of the surgical procedure, with the use of a single portable instrument. Most advanced FGS are based on near infrared (NIR) light that has proven its value for multiple surgical procedures. (6) The advantages of NIR light (wavelengths of 700-900 nm) are (1) avoiding tissue autofluorescence which emits in the visible spectrum (400-550 nm) and (2) a relatively high tissue penetration capacity (5-10 mm), which enables detection of targets even if they are covered by blood or a thin layer of tissue.

The challenge for this technology is developing tumor-specific fluorescence imaging probes to target cancerous tissue. The first-in-human proof of principle of using FGS tumor-specific probe was provided in 2011. Tumor-specific fluorescence imaging of the folate receptor a using folate-FITC, was proven to be a specific and sensitive method for real-time identification of peritoneal carcinomatosis during surgery in patients with ovarian cancer (7). Later, the therapeutic antibody bevacizumab that binds to VEGF-A, an angiogenesis receptor, was conjugated to the NIR fluorophore IRDye800CW and showed to be efficient for intraoperative identification of breast cancer (8). Since then, only a few tumor-targeted techniques to visualize tumor cells using fluorescence labelled ligands have been described in in vivo studies in adult patients for different types of tumors (9, 10). So far, in pediatric oncology the use of targeted-fluorescent probes have not been implemented.

### Glycodisiaganglioside 2 (GD2)

GD2 is part of a family of gangliosides containing 2 N-Acetylneuraminic acids (11). GD2 is overexpressed by neuroblastomas, most melanomas and some other tumors (12). In normal human tissues, GD2 expression is expressed on neurons, skin melanocytes, and peripheral sensory nerve fibers (13). Immunotherapy with dinutuximab has shown to improve event-free and overall survival among children with high-risk neuroblastoma (14). Currently, in Europe a similar product has been approved by EMA for the treatment of neuroblastoma, Dinutuximab-beta or Qarziba®, which is authorized for the treatment of high-risk neuroblastoma in patients aged 12 months and above. In high risk treatment of neuroblastoma immunotherapy with the Qarziba, targeting the tumor-associated antigen disialoganglioside GD2 is now part of the standard therapy. IRDye800CW has previously been conjugated to other immunotherapy antibodies, such as cetuximab and bevacizumab without altering their binding capacity and/or toxicity (15, 16). In our preclinical studies, we developed and validated a clinical translatable GD2-specific NIR fluorescent imaging probe that can be used to visualize Neuroblastoma: Dinutuximab-beta-IRDye800CW aka anti-GD2-IRDye800CW. This study will be the first to determine the recommended dose for anti-GD2-IRDye800CW in pediatric patients with neuroblastoma.

### 2.2 Background Therapeutic Information

### Anti-GD2

Anti-GD2, or Dinutuximab-Beta is a chimeric (human-murine) IgG1K monoclonal anti-GD2 antibody similar to Dinutuximab, the anti-GD2 immunotherapeutic is known to improve the survival of high-risk neuroblastoma (14). Its molecular weight is approximately 148 kDa. Anti-GD2 binds to cell surface glycolipid GD2 expressed on many neuroblastoma cells as well as on many normal cells in the central nervous system and peripheral nerves and induces lysis of the GD2-expressing cells. The supposed mechanisms of cell lysis are antibody-dependent cell-mediated cytotoxicity (ADCC) and complement-dependent cytotoxicity (CDC).

### IRDye-800CW

Anti-GD2 is in our study conjugated to a dye, IRDye 800CW, which is a near-infrared (NIR) fluorophore. IRDye800CW has an absorption and emission wavelength in the NIR spectrum, at 778 nm and 794 nm, respectively. Preclinical studies proved IRDye800CW is not immunogenic and the no adverse effect level (NOAEL) according to rats is 20 mg/kg (15). The NHS ester of the dye reacts with a primary aliphatic amine of the antibody, thereby labeling the antibody through an amide bond.

### 2.3 Correlative Studies Background

### Preclinical Data

In preclinical work our group (23) has evaluated the NIR fluorescence tracer anti-GD2-IRDye800CW in an in vivo mouse model. First, a dose escalating cohort study was performed. Because the TBR was optimal at a dose of 1.0 nmol, this was chosen for further examinations. The human-derived relapsed neuroblastoma cell line KCNR, known to be overexpressing GD2, was subcutaneously transplanted into immunocompromised mice. After intravenous tail injection of 1.0 nmol anti-GD2-IRDye800CW, fluorescence was measured in different tumors on mouse models using the PEARL in vivo imager and the QUEST fluorescence near-infrared camera system. Tumor tissue was clearly identified in mouse models. Optimal TBR was obtained after 96 hours. Microscopic evaluation using the fluorescence microscopy identified a strong fluorescence signal in the tumor, with clear demarcation of tumor tissue (23).

### 3. Patient Selection

### 3.1 Subject Inclusion Criteria

Patients with the diagnosis of neuroblastoma as defined by histopathology (confirmed by the PMC Department of Pathology), who will be operated for NB as standard of care procedure
Patients older than 1 year of age and not older than 18 years.
Patients that received prior treatment with antibodies must have a HACA antibody titer which is undetectable with our detection method using mass spectometry. This to confirm that the patient is not forming antibodies against the immunotherapy antibody treatment. Human anti-mouse antibody positivity is allowed.
Signed informed consent indicating awareness of the investigational nature of this program.
   absence of any psychological, familial, sociological or geographical condition potentially hampering compliance with the study protocol and follow-up schedule; those conditions should be discussed with the patient before registration in the trial
   before patient registration, written informed consent must be given according to ICH/GCP, and local regulations.

### Exclusion Criteria

### Presence of Grade 3 Toxicity of Previous Dinutuximab-Beta Treatment Prior to Surgery

### 4. Registration Procedures

Eligible patients will be entered on study centrally at the Princes Máxima Center by the Study Coordinator. Following registration, patients should begin protocol treatment dependent on the date of the scheduled surgery. Issues that would cause treatment delays should be discussed with the Principal Investigator. If a patient does not receive protocol therapy following registration, the patient's registration on the study may be canceled. The Study Coordinator should be notified of cancellations as soon as possible.

To register a patient, the following documents should be completed by the Study

### Coordinator:

Copy of required laboratory tests
Signed patient consent form

The Study Coordinator will then verify eligibility. To complete the registration process, the Coordinator will
assign a patient study number
register the patient on the study
assign the patient a dose
call the research nurse and data manager and verbally confirm registration.

### 5. Trial Design

This is a non-randomized, open label dose escalation phase I/II study in neuroblastoma patients, consisting an initial pharmacokinetic (PK) study of anti-GD2-IRDye800CW in patients with neuroblastoma undergoing surgery. The aim is to establish a safe and effective dose of anti-GD2-IRDye800CW for intra-operative detection of neuroblastoma using near-infrared fluorescence. Based on PK data and the TBR (ex vivo>2.0), an optimal dose will be determined.

Since this is a phase I/II, first-in-child, study, we will use a rolling 6 design. Other than to test the efficiency of the fluorescence, we have not currently planned further dose escalation steps.

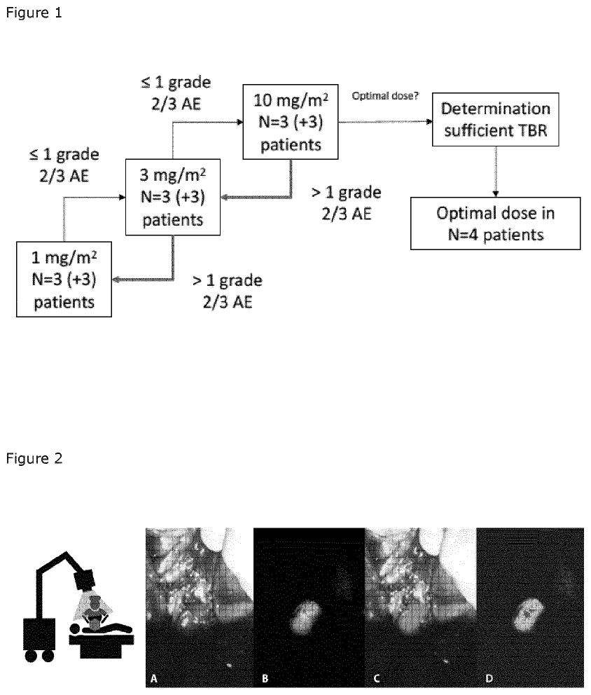

Our main endpoint is to achieve safe and effective imaging. Instead, we will act by an adaptive recruitment strategy (FIG. 1). Dose limiting toxicity (DLT) will be studied, but we do not expect any new adverse effects since the maximum dose will be equivalent to the dose that is currently used for 24 hours of immunotherapy, compared to 240 hours of immunotherapy in a therapeutic setting. Therefore the Maximum Tolerated Dose which has been defined for anti-GD2 treatment will not be reached and not be assessed in this study (17, 18).

### 5.1 Dosing/Imaging Schedule

### Rationale for Starting Dose

Since we are looking for an effective, but above all safe dose with the least toxicity as possible, the optimal biologic dose of anti-GD2-IRDye800CW will be determined by scaling up dosages after starting with a microdose as advised in literature and by the Food and Drug Authority [24]. A microdose is defined as max. 1% of the therapeutic dose. This will be 1 mg/m² in 3 patients. If tolerable, we will continue using dosages of 3 mg/m² and 10 mg/m² in groups of 3 patients.

A preclinical study in our institute in a mouse model with an injected anti-GD2-IRDye800CW dose of 1.0 nmol showed that an optimal TBR was reached after 96 hours (23), after which the signal in the tumor remained high in comparison to background. The human equivalent dose (HED) of this dose is 22 mg/m², which is above the dose of 10 mg/m² that is currently given per 24 h and above the 20 mg/m² that is reported to have minimal toxicity in children (20) and needs intensive supportive care.

However, according to our study results, a dose of 0.3 nmol in mice (0.045 mg anti-GD2-IRDye800CW) should still generate a TBR>2.0 at 96 hours after infusion. The HED of this dose is 6.75 mg/m² in children, which is higher than our starting (micro) dose, but lower compared to our end dose. Therefore, we expect to be able to determine the most optimal dose within this dosage range. Also, in the current Dinutuximab-beta treatment clinical trials with comparable antibodies, the injected dose is 10 mg/m² per cycle and is feasible when supportive care is given (25).

To follow the protocol of the immunotherapy schedule, infusion of the antibody will be 10 mg/m² per 24 hours. Approximately 96 hours after start of the infusion, the surgery will take place. We expect the time of imaging to be similar to our preclinical experiments. The time of administration for similar size antibodies in other fluorescence guided surgery literature usually correlated to the preclinical experiments (7, 21). However, if we see no fluorescence signal with the highest dose, we have to reconsider our administration time point before surgery.

| | Dose of anti-GD2- |
|---|---|
| Dose level | IRDye 800 CW |
| X | 1 mg/m² |
| X + 1 | 3 mg/m² |
| X + 2 | 10 mg/m² |
| RP2D | 10 or 3 or 1 mg/m² |

In addition, the clinical trial might be modified to include higher doses of anti-GD2-IRDye 800 CW administered at the optimal time point before tumour detection in one or more new cohorts of patients, such as 11, 12, 13, 14, 15, 16, 17, 18, 19 or 20 mg/m² body surface area. Similar considerations apply evaluating DLT and adverse events in patients administered such higher doses.

### Imaging Schedule

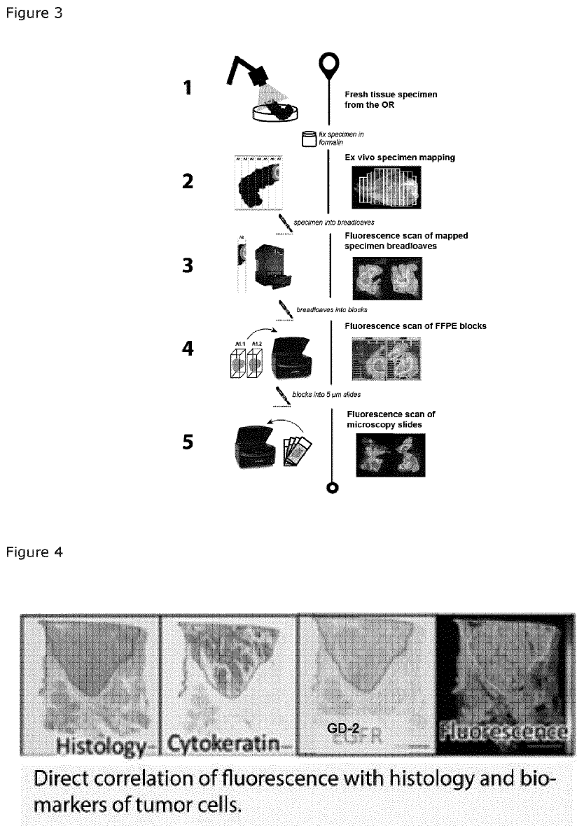

During surgery and during pathology examination, there will be multiple time points where the fluorescence signal will be measured and afterwards analyzed (See section 6.2, FIG. 3).

### Rolling 6 Strategy

The starting dose of anti-GD2-IRDye800CW will be 1 mg/m² (see section 5.1). This is a microdose and lower than the minimal toxicity dose for Dinutuximab (20 mg/m²). Since this is a proof-of-principle study, first to be ever performed in children, we have chosen to test three different set doses in groups of a maximum of three-six patients. The sample size of this feasibility study will be 12-22 patients based on the Rolling-6 design for the safety part plus an expansion cohort treated at the recommended dose including the patients that are enrolled at that dose level in the safety part. This means that 12 patients will be included in case of adverse effects in dosage group 3 mg/m², (namely 6 patients in dosage group 1 mg/m², 2 patients in dosage group 3 mg/m² and an expansion cohort of 4 patients in dosage group 1 mg/m²). If there is no limitation by adverse effects, 22 patients will be included.

Dose-escalation will be based on AEs (FIG. 1), different from the common side effects (frequency≥ 1/10) as can be expected from dinutuximab-alone infusion as defined by the manufacturer in the product information provided by the EMA [26].

### 5.1.1 Recommended Phase II Dose

The recommended Phase II (RP2D) dose will be the lowest dose where ≥5/6 patients in the cohort have a mean ex vivo TBR>2.0 (see section 5.2). If the groups are alike, the lowest dose with the TBR>2.0 will be chosen as the RP2D.

### 5.2 Methods and Endpoints

### Analysis of Primary Endpoints

Treatment related new adverse events after injection of the conjugate anti-GD2-IRDye800CW not to be expected from dinutuximab-alone infusion.
Tumor to background ratio (TBR) of at least 2.0 ex vivo and sufficient for detection during surgery. TBR is established by dividing the mean fluorescence signal of the tumor by the mean fluorescence signal of the background. Background is determined by fluorescence intensity in healthy kidney, liver and surrounding background tissue.

### Analysis of Secondary Endpoints

Changes in serum pharmacokinetics; blood samples will be taken at t=0 (just before infusion), t=24 h (after infusion), t=72 h (after infusion), t=96 h (immediately before surgery), t=120 h (24 h after surgery) and t=144 h (48 h after surgery).
The sensitivity and specificity of our novel fluorescent agent to detect GD2-expressing, vital tumor cells. Pathologic status of removed fluorescent tissue and of removed nonfluorescent tissue will be compared to fluorescent status. After surgery, the ex vivo resected specimen fluorescence hotspots will be marked within the same day. Pathologic analysis based on morphological characteristics, between 1-10 days after surgery, will differentiate between different neuroblastoma stages and/or necrosis. The pathology morphology report of these samples will be compared with the mean fluorescence intensity.
Standard operating procedure for ex vivo processing of surgical specimen will be performed (see extensive imaging plan at 6.2). Upon excision during surgery, the entire fresh specimen will be cut into slices and imaged by the optical imaging system in overlay images of white light and fluorescence mode. Next, tissue will be embedded into paraffin blocks and overlay images of white light and fluorescence will be taken. H&E staining and GD2 expression will be performed and the morphology of the sections will be assessed by an experienced pediatric oncology pathologist according to the INPC classification.

### 6. Treatment and Imaging Plan

### 6.1 Treatment Administration

Treatment will be administered on an inpatient basis. After the infusion ends, and if the patients is pain-free for 1 hour, the patient can be discharged from the hospital and return 1 day prior to surgery (as in standard care). Reported adverse events and potential risks are described in Section 7. No investigational or commercial agents or therapies other than those described below may be administered with the intent to treat the patient's malignancy.

Pain and allergic reactions are clinically significant in the short term, since GD2 is expressed in healthy tissue on pain fibers. To limit the side-effects of the anti-GD2 (pain, urticaria), patients shall receive opiates and/or antihistamines similar to standard immunotherapy treatment protocol.

| | Regimen Description | | | | |
|---|---|---|---|---|---|
| | Premedications; | | | | |
| Agent | Precautions | Dose | Route | Infusion time | Schedule |
| Anti-GD2- | Administer | ** in 500 cc | IV | 10 mg/m2 over 24 hours, | 96 hours |
| IRDye800CW | adequate opioid | NS | (10 mg/m2 over | infusion 2 ml/h | before |
| | dose and antihistamine | | 24 hours, | (lower doses in same rate, | surgery |
| | medication starting | | infusion) | less infusion time, so | |
| | 1 hour prior to anti- | | | 0.42 mg/m2 per hour) | |
| | GD2-IRDye800CW | | | | |

** Doses as appropriate for assigned dose level.

### 6.2 Acquisition of Imaging Data

Here we present a method for reporting results from fluorescence-guided oncologic surgery studies as suggested in literature [27]. This method is based on the requirement from all phases of the clinical trial; intraoperative imaging, ex vivo imaging, and pathologic assessment to assess the efficacy and accuracy of tumor-specific imaging agents.

### Intra-Operative Imaging

Intra-operative imaging timepoints:
1. After exposure of the tumor and prior to surgical excision
2. If needed, after partly excision of the tumor
3. After resection of the tumor
4. After exposure of lymph nodes (before resection)

During these specific timepoints we will report on the intra-operative imaging results (see FIG. 2) presenting a bright field image (A), a black-and-white fluorescent image (B), fluorescent overlay image (C), and a fluorescent heat-map (D), in this case of a tumor-positive lymph node during abdominal surgery.

### Ex Vivo Back-Table Imaging

After removal of the specimen from the patient, the fresh specimen is imaged at the backtable, using the surgical camera. This data will be presented in the same manner as the intra-operative imaging.

After imaging fresh surgical specimen, the specimen is formalin fixed before the specimen is processed into breadloaves. Formalin fixed tissue can still be used for qualitative and quantitative analysis of fluorescence imaging signal, compared to paraffin embedded tissue which is only suitable for qualitative data analysis.

### Histopathologic Correlation

To determine whether the novel imaging agent targets the tumor and the GD-2 positive cells, we will ensure exact correlation of the surgical specimen with pathology assessment.

This will be done using a mapping process of the ex vivo specimen, as illustrated in FIG. 3. During step 1 the fresh tissue specimen is imaged ex vivo to ensure images of all angles using the Quest camera. After formalin fixation, the specimen is mapped and sliced into breadloaves in step 2. Once the breadloaves are generated fluorescence imaging using the Quest camera is performed of the breadloaves separately. In step 4 cassettes are made and imaged either on a fluorescence scanner (Odyssey). The cassettes are paraffin embedded and cut into microscopy slides. In step 5, these slides are used for pathology assessment and fluorescence scanning (Odyssey).

### Confirmation of Tumor Targeting

As discussed above, this step of the process allows for the direct correlation between tissue fluorescence and actual tumor status on H&E. In early phase trials, this step is critical to ensure the novel agent is indeed targeting the tumor. Confirmation of tumor status on the H&E slides should be performed in close collaboration with a trained pathologist, who is preferably blinded for the fluorescence imaging results. With the use of a fluorescence scanner, the location of fluorescence can be detected with a μm resolution.

If the mapping process, as described above, is performed in a rigorous manner, the correlation generated at this step could be reasoned backward to the fluorescence imaging results of the fresh entire specimen acquired at back table imaging, and sometimes even to the intra-operative findings. However, this last step remains challenging due to difference in orientation, and the influence of ambient light on fluorescence.

### Confirmation of Molecular Targeting

In case of a targeted agent, the confirmation of molecular targeting of the imaging agent should be a required step of the data analysis. Here, co-localization of the target of interest and fluorescent signal shows that the agent actually targets the biomarker, compared to fluorescence in the tumor based on EPR effect. Co-localization can be performed using sequential slides of which one is used for immunohistochemistry (IHC) for the target and one for fluorescence scanning, as illustrated in FIG. 4. Unfortunately, due to processing of slides for IHC, one cannot use the same slide for this co-localization since the fluorescent signal is lost during the IHC process. However, this can be done when only performing an H&E staining.

In addition to IHC for the target of interest, some additional staining could be of interest during this part of data analysis for better understanding of the behavior of the agent, for example for markers that influence delivery of the agent, such as vascular density, presence of lymphatic vessels, and amount of proliferation cells. Therefore, this staining will uniquely be performed routinely during early phase trials when this information is relevant to advancing the agent. In later phase trials, one could choose to perform additional staining if the imaging results of a patient are out of the range of what is expected.

### Analysis

The fluorescence intensity of all images will be quantified measuring the mean fluorescence intensity (MFI) of the Quest Artemis Imaging System (Quest Medical Imaging, Middenmeer, The Netherlands) and the corresponding software. The MFI of the tumor and the MFI of the background area will be calculated. Also, the MFI of the kidney and liver will be collected. To get the TBR, the MFI of the tumor will be divided by the MFI of the background.

### 6.3 Support Supportive Care Guidelines

The supportive care guidelines will be carried out as described in the long-term infusion protocol for immunotherapy:

A pediatric oncologist, specialized nurse practitioner and a nurse specialist will be involved during the infusion. Pain and allergic reactions are clinically significant in the short term, since GD2 is expressed in healthy tissue on pain fibers. It is thought that complement activity is primarily responsible for pain side effects (22). To limit the side-effects of the anti-GD2 (pain, urticaria), patients will receive analgesia and antihistamines according to standard treatment protocol starting 1 hour before infusion of anti-GD2.

Standard supportive care:
Paracetamol oral, 60 mg/kg/day.
Morfine
levocetirizine oraal 2 dd, dose based on age

Children will be monitored during infusion. Blood pressure and heart rate are to be assessed every 30 min for the first 2 hours, after that every 5 hours during the 24-hour infusion. Monitoring will be similar as described in the long-term infusion protocol for dinutuximab (see appendix 1).

### 6.4 Dosing Delays/Dose Modifications and Adverse Event Management

The infusion will be stopped in case of any severe adverse events, graded according to the CTCAE as ≥grade 3, not to be expected from dinutuximab-alone infusion, or upon the opinion of the treating physician. The amount of anti-GD2-IRDye800CW administered at that point, will be calculated and documented. Dose adjustments are to be made according to the greatest degree of toxicity, but are not to be expected giving the results of the anti-GD2 immunotherapy protocol (see 6.6). Adverse events will be graded using the NCI Common Terminology Criteria for Adverse Events Version 5.0 (CTCAE).

Adverse events known from dinutuximab-alone infusion studies are:
Cytokine release syndrome (fever, hypotension, urticaria)
Anaphylactic reaction
Capillary leak syndrome (hypotension, tachycardia, oedema)
Decreased diuresis
Bladder retention (as a side effect from the morfine)
Visual problems (In case of less sight: discuss with the oncologist)
Peripheral neuropathy, transverse myelitis, unknown neurological complaints
Systemic infections
Haematological toxicity
Liver- and electrolyte irregularities

These toxicities are merely present at administration of higher doses of Dinutuximab than 10 mg/m²/day.

### 6.5 Duration of Follow Up

Only one IV injection will be given. Patients will be observed for safety during a two-week period post surgery (EOT visit). The half-life of the product is 8 days, and therefore we consider this sufficient observation time.

6.6 Criteria for Removal from Study

### Adverse Events

In case of severe new adverse events during administration, the infusion of the anti-GD2-IRDye800CW will be stopped. The dose that is administrated will be calculated after the stop of iv infusion. Since the patients will not receive a second dose, the patients will not be removed from the protocol. During surgery, it will be investigated if the amount of dose that reached the patient is sufficient for imaging the tumor. Patients with adverse events will be treated and followed according to established, acceptable medical practice. The adverse events and the adjusted dose must be documented in the Case Report Form.

### Withdrawal of Consent

The patient's desire to withdraw from the study may occur at any time. The investigator should carefully consider whether the patient's withdrawal of consent is due to an adverse event, and if so, record the adverse event as the reason for withdrawal.

Withdrawal by the physician for clinical reasons not related to study drug treatment in the absence of an adverse event.

### 6.7 Anti-Drug-Antibody Development Against Anti-GD2-IRDye800CW

### Anti-Drug Antibody Formation Against Anti-GD2-IRDye800CW

In the current study we will evaluate the possible formation of anti-drug antibodies (ADAs) resulting from the administration of anti-GD2-IRDye800CW used for intra-operative detection of neuroblastoma with near-infrared fluorescence. ADA formation is of particular concern during monoclonal antibodies (mAbs) therapy due to the potential immunogenicity of the mAb. This immune system mediated reaction could potentially result in loss of response and adverse events, which consequently could result in treatment failure. ADA development is strongly dependent on multiple factors, for instance; the structural origin of the mAb, e.g. human, humanized or non-human, the dosage of the mAb, structural size of the mAb and co-medication like immunosuppressive agents. ADA is a broader term which can be divided in antibodies which develop against mAbs based on their origin, such as human anti-chimeric antibodies (HACA), human anti-humanized antibodies (HAHA), and human anti-mouse antibodies (HAMA) (28).

As stated in the summary of product characteristics (SmPC) of DNX, the development of ADAs is a well-known phenomenon which could develop during monoclonal chimeric antibodies therapy. In previous studies ADA concentrations were detectable in up to 62% of the studied patients (n=105) after standard dosing regimens (26).

In this current study, only 10 mg/m² of DNX-IRDye800CW, a fraction of the therapeutic dosage, ca. 100 mg/m² of DNX, is administered before surgery. DNX is however a chimeric mAb which has more immunogenic potential in comparison to a human or humanized mAb. Besides, the possible additional immunogenicity of the added IRDye800CW to DNX, or to any mAb for that manner, has never before been studied. Previous studies using this fluorescent technique with bevacizumab and cetuximab have not looked into any additional immunogenicity (28).

### Analytic Methods for Detecting ADAs Against DNX

During registration and follow-up studies for DNX, analytic methods have been developed to measure ADA concentrations against DNX. Generally an enzyme-linked immunosorbent assay (ELISA) technique is used, this analytic method uses a binding antigen to create a complex with the desired antibody, a reporter is linked to create a signal which can be measured in order to quantify to amount bound to the binding agent. The ADA assay was developed using the anti-Id mAb ganglidiomab, which specifically binds DNX. Ganglidiomab is used as a capture mAb (binding antigen) for the assay in order to mimic the binding of an ADA to DNX. Although this ADA ELISA method has been previously validated and widely used, concerns have been raised based on its results (25).

In the studies two different ELISA methods are used, one designed to measure DNX and a separate assay for ADA levels. The EMA published an assessment report in 2017 in which they concluded that major issues have been shown in the assay concerning drug-ADA interaction. The ADA assay seems to have poor drug tolerance, which means that it is difficult for the assay to detect ADAs in the presence of detectable DNX levels because DNX might interfere with the assay. Due to this possible interference in both assays, the immunogenicity data is not considered reliable. The unreliability of the current assays could explain the variability between ADA levels in the different studies (25).

Furthermore, there are two types of ADA which can develop; neutralizing antibodies (NAb) and non-neutralizing antibodies. NAbs are clinically the most relevant to the treatment, these ADAs actively block the pharmacological function of the drug. The current analytic ELISA method is not able to differentiate between the types of ADA that develop (30, 35).

### Results of ADA Studies to DNX

Although the exact effect of ADA on the pharmacokinetics of DNX has not been fully clarified, previous studies have shown an increase of +37% of the volume of distribution (Vd). This increase in Vd due to ADA formation could be translated to approximately a <10% decrease in DNX exposure within the first 24 hours after administration (25, 26).

In 2017 the EMA reviewed in an assessment report the immunogenicity of DNX on the basis of multiple studies. The studies found that the majority of patients developed ADAs to DNX, between 56% and 73%. One study found no positive ADA patients (n=32), which may result from the fact that these patients underwent hematopoietic stem cell transplantation before DNX treatment resulting in B-cell depletion and thus insufficient immune response (25).

Siebert et al. 2016 studied the PK/PD of DNX and ADA in neuroblastoma patients treated with therapeutic DNX, 100 mg/m2+isotretinoin. In 19% of analyzed patients (n=53) ADA levels were detected using their ELISA method at the end of cycle 5 of the treatment but already detected a reduced mAb concentration in cycle 2 in patients whom would become ADA positive. Development of ADA resulted in reduction of DNX levels in subsequent treatment cycles in ADA-positive patients (cycles 2, 3, 4 and 5) compared to ADA-negative patients; reduced and almost complete lack of administered DNX was observed in ADA-positive responders. In addition a significant reduction in complement-dependent cytotoxicity due to DNX was found in ADA positive patients compare to ADA negative (35).

More recently Yu et al. 2021, evaluated a long term phase III follow-up study of 226 children treated with isotretinoin with or without DNX. In this study they used an ELISA method to measure ADAs. Of the 122 patients treated with DNX, 13 developed ADAs over the course of the study. No ADAs were detectable after cycle 1 but were detectable before cycle 3. Detectable ADAs against DNX were associated with lower through and peak levels during cycle 4 and 5. However, the patients whom developed ADAs demonstrated in vivo a neutralizing effect on detectable DNX (31).

### New ADA Assay Development

Given the limitations of the bioanalytical methods, data are currently insufficient to properly evaluate the impact of the formation of anti-drug antibodies on pharmacokinetic and pharmacodynamics parameters, as well as on the efficacy and safety of DNX (25). As shown in previous studies by the research group performing these assays (Alwin Huitema), liquid chromatography-tandem mass spectrometry (LC-MS/MS) has proven to be a reliable bioanalytical platform for both small and large molecule quantification. LC-MS/MS offers multiple advantages compared to other techniques in terms of selectivity, method development time, increased precision due to the use of internal standards and multiplexing capabilities. Our research group has already demonstrated the use of LC-MS/MS in order to reliably measure ADAs to multiple mAbs and even differentiate the type of ADA in order to solely measure Nabs (30). In addition, we already developed and published a robust, validated and reliable total DNX quantification method using the LC-MS/MS. In this method selectivity, stability, and matrix effect were in concordance with EMA guidelines (34).

By using the LC-MS/MS and the previous described concept of utilizing ganglidiomab as a capture mAb to mimic ADA binding for validation, it is possible to develop a new, more sensitive and precise method for measuring ADAs to DNX. Measuring ADA to DNX with the LC-MS/MS would nullify the concerns raised by the EMA for the current analytic techniques and produce reliable and accurate ADA and drug plasma levels for pharmacokinetics (PK) and pharmacodynamics (PD) evaluation.

### Conclusion

In our study where we administrate DNX-IRDye800CW in a fraction of the normally therapeutic dose of DNX it is theoretically unlikely that ADA formation will occur after the first cycle. However, uncertainties concerning the current analytic methods used in previous studies make it hard to make a reliable statement about ADA and drug levels resulting in inadequate PK and PD evaluation and thus impossible to make certain conclusions. Possible ADA formation due to DNX-IRDye800CW administration could harm the successive treatment with either therapeutic DNX cycles or interfere with a potential second DNX-IRDye800CW administration before a second operation by neutralizing DNX as shown in previous studies (31, 35).

Chosen sampling moments for DNX in the study are in coherence with the time points at which the patient has to be in the hospital, and thus won't cause additional burden for the patient;
Baseline (before infusion)
24 h (end of infusie)
72 h (day before surgery)
96 h (surgery)
120 h (1 day postoperative)
144 h (2 days postoperative)

### 7. Adverse Event Reporting Requirements

Any serious adverse event must be reported to the METC as soon as possible but no later than 5 calendar days.

### 7.1 Expected Adverse Events Using Anti-GD2-IRDye800CW

This is a first-in-human study of the conjugate anti-GD2-IRDye800CW. Separately, anti-GD2 and IRDye800CW are cGMP products and have been clinically tested and approved for human use. Besides that, anti-GD2-IRDye800CW will be used in a low dose, compared to clinical use of dinutuximab-alone. Therefore, we do not expect any new adverse events other than reported for Dinutuximab (anti-GD2). However, we will monitor closely for adverse events.

### 7.2 Adverse Event Characteristics

CTCAE term (AE description) and grade: The descriptions and grading scales found in the revised NCI Common Terminology Criteria for Adverse Events (CTCAE) version 5.0 will be utilized for AE reporting. All appropriate treatment areas should have access to a copy of the CTCAE version 5.0. A copy of the CTCAE version 5.0 can be downloaded from the CTEP web site http://ctep.cancer.gov/protocolDevelopment/electronic_applications/ctc.htm.
For expedited reporting purposes only:
   AEs for the agent that do not require expedited reporting are defined in Section 7.1.
   Other AEs for the protocol that do not require expedited reporting are outlined in section 7.3.
Attribution of the AE:
   Definite—The AE is clearly related to the study treatment.
   Probable—The AE is likely related to the study treatment.
   Possible—The AE may be related to the study treatment.
   Unlikely—The AE is doubtfully related to the study treatment.
   Unrelated—The AE is clearly NOT related to the study treatment.
An adverse event is considered serious (SAE) if it results in ANY of the following outcomes:
   Death
   A life-threatening adverse event
   An adverse event results in inpatient hospitalization or prolongation of existing hospitalization for ≥24 hours
   A persistent or significant incapacity or substantial disruption of the ability to conduct normal life functions
   A congenital anomaly/birth defect
   Important Medical Events (IME) that may not result in death, be life threatening, or require hospitalization may be considered serious when, based upon medical judgment, they may jeopardize the patient or subject and may require medical or surgical intervention to prevent one of the outcomes listed in this definition.

### 7.3 Routine Adverse Event Reporting

All Adverse Events must be reported in routine study data submissions.

### 8. Pharmaceutical and Imaging Agent Information

A list of the adverse events and potential risks associated with the investigational agent administered in this study can be found in Section 6.4.

Source and Pharmacology: Anti-GD2 is a chimeric monoclonal antibody of the IgG1 subclass. It is purified by affinity and ion exchange chromatographines and filtered to remove pathogens, included viruses. The final product is tested to assure that it is free of nucleic acid, viruses, bacteria, fungi, mycoplasm and pyrogens.

Supplier: Anti-GD2-IRDye800CW will be manufactured under GMP conditions by EUSA Pharma and consequently conjugated to IRDYe800CW by a contracted manufacturer (GMP pharmacy Leiden University Medical Center) as an investigational agent for the Princess Maxima Center.

Route of administration: IV infusion

### 8.1 Investigational Agent

### Dinutuximab-Beta

Anti-GD2, or Dinutuximab-Beta is a chimeric (human-murine) IgG1K monoclonal anti-GD2 antibody similar to Dinutuximab, the anti-GD2 immunotherapeuticum known to improve the survival of high-risk neuroblastoma (14). Its molecular weight is approximately 148 kDa. Anti-GD2 binds to cell surface glycolipid GD2 expressed on many neuroblastoma cells as well as on many normal cells in the central nervous system and peripheral nerves and induces lysis of the GD2-expressing cells. The supposed mechanisms of cell lysis are antibody-dependent cell-mediated cytotoxicity (ADCC) and complement-dependent cytotoxicity (CDC).

### IRDye-800CW

Anti-GD2 is in our study conjugated to a dye, IRDye 800CW, which is a near-infrared (NIR) fluorophore. IRDye800CW has an absorption and emission wavelength in the NIR spectrum, at 778 nm and 794 nm, respectively. Preclinical studies proved IRDye800CW is not immunogenic and the no adverse effect level (NOAEL) according to rats is 20 mg/kg (15). The NHS ester of the dye reacts with a primary aliphatic amine of the antibody, thereby labeling the antibody through an amide bond.

### Combination of Dinutuximab-Beta and IRDye-800CW

IRDye800CW has previously been conjugated to other immunotherapy antibodies, such as cetuximab and bevacizumab without altering their binding capacity and/or toxicity (15, 16). In our preclinical studies, we developed and validated a clinical translatable GD2-specific NIR fluorescent imaging probe that can be used to visualize Neuroblastoma: Dinutuximab-beta-IRDye800CW aka anti-GD2-IRDye800CW (23). We evaluated the NIR fluorescence tracer anti-GD2-IRDye800CW in an in vivo xenograft mouse model. First, a dose escalating cohort study was performed. Because the TBR was optimal at a dose of 1.0 nmol, this was chosen for further examinations. The human-derived relapsed neuroblastoma cell line KCNR, known to be overexpressing GD2, was subcutaneously transplanted into immunocompromised mice. After intravenous tail injection of 1.0 nmol anti-GD2-IRDye800CW, fluorescence was measured in different tumors on mouse models using the PEARL in vivo imager and the QUEST fluorescence near-infrared camera system. Tumor tissue was clearly identified in mouse models. Optimal TBR was obtained after 96 hours. Microscopic evaluation using the fluorescence microscopy identified a strong fluorescence signal in the tumor, with clear demarcation of tumor tissue (23). This study will be the first to determine the recommended dose for anti-GD2-IRDye800CW in pediatric patients with neuroblastoma.

### 9. Study Calendar

### 9.1 Baseline/Pre-Study Evaluations

Visit 1: Screening, Day −28 to day 0

During Visit 1/Screening (up to 4 weeks prior to surgery), subjects who provide written informed consent, and who have an established diagnosis of neuroblastoma will undergo.
Complete medical history and physical examination;
Vital signs and weight;
Clinical chemistry and hematology assessments;
Evaluation of inclusion and exclusion criteria;
Collection of concomitant medication information.

This population of patients can consist of patients who already underwent a preoperative biopsy.

### 9.2 During Treatment

Visit 2:96-72 hours prior to surgery

Eligible subjects will have the following procedures performed:
Any changes in concomitant medications will be recorded and AEs will be collected. Medical history and physical examination will be updated as needed. During and post-infusion of the anti-GD2-IRDye800CW, AEs and ADEs will be collected.
A single dose of anti-GD2-IRDye800CW will be administered IV (over 24 hours or less) to each subject starting 96 hours prior to surgery. The dose will be assigned by the Trial Pharmacy.
Vital signs will be taken every 1 hour during infusion for the first 2 hours followed by every 5 hours until discharge from hospital. It will be followed up daily from the moment the patient is again admitted to the hospital until surgery is initiated.
Blood samples will be taken at t=0 (just before infusion), t=24 h (after infusion), t=72 h (after infusion), t=96 h (immediately before surgery), t=120 h (24 h after surgery) and t=144 h (48 h after surgery). A time range of +/−1 hour is approved.

Visit 3: Time of surgery
AE's will be collected 24 hours before surgery
Vital signs will be taken prior to surgery

### 9.3 Follow-Up Evaluation

Visit 4 (Follow-up, Day 14+3 after surgery, this can be planned together with the follow-up appointment to the clinician)

Subjects will return to the site and the following information will be recorded:
Vital signs;
AE assessments;
Concomitant medications;
Blood samples for clinical laboratory assessments and serum anti-GD2-IRDye800CW level

### 9.4 Summary Table

All indicated times are in a time range of +/−1 hour. The exact timing of the measurement should be written on the CRF.

TABLE 1
Schedule of study procedures

| | | | | | | | | | +14 days |
|---|---|---|---|---|---|---|---|---|---|
| | Pre- | | | | | | | | after |
| | Study | Baseline | Dose | +24 h | +72 h | +96 h | +120 h | +144 h | surgery |
| Anti-GD2- | | | X | | | | | | |
| IRDye800CW | | | | | | | | | |
| Informed consent | X | | | | | | | | |
| Demographics | X | | | | | | | | |
| Medical history | X | | | | | | | | |
| Concurrent meds | | | | X-------------------------- X | | | | | |
| Physical exam | X | X | | | | X | | | X |
| Vital signs | X | X | X | X | X | X | X | X | X |
| Height | X | | | | | | | | |
| Weight | X | X | | | | | | | X |
| Performance status | X | | | | | | | | |
| Serum chemistry^{a} | X | X | X | X | X | X | X | X | X |
| Serum chemistry^{b} | | X | | | | X | | | |
| ADA measurements | | X | X | X | X | X | X | X | |
| Serum Dinutuximab | | X | X | X | X | X | X | X | X |
| levels | | | | | | | | | |
| Adverse event | | | | X-------------------------- X | | | | | |
| evaluation | | | | | | | | | |
| Surgery and | | | | | | X | | | |
| fluorescent | | | | | | | | | |
| imaging | | | | | | | | | |

^{a}Hemoglobine, thrombocytes, alkaline phosphatase, total bilirubin, BUN, creatinine, calcium, chloride, sodium, potassium, SGOT [AST], SGPT [ALT], glucose, LDH.
^{b}Albumin, INR, PT, phosphorus, total protein.

### 10. Statistical Considerations

### 10.1 Study Design/Endpoints

### Result Definitions:

| | |
|---|---|
| Tumor/ signal-to-background | Background is determined by |
| ratio | fluorescence intensity of normal tissue |
| Pathologic status of removed | During surgery and ex-vivo, on |
| fluorescent lesions and of | resected specimen, fluorescence |
| removed non-fluorescent lesions | hotspots will be marked. All resected |
| | specimen will undergo pathologic |
| | analysis to define tumor involvement. |
| Sensitivity | Proportion of GD2 positive lesions |
| | which are also fluorescent. |
| Positive predictive value | Proportion fluorescent lesions that |
| | contain GD2 expressing tumor cells |
| Specificity | Proportion of non-malignant tissue that |
| | is not fluorescent (and GD2 negative) |

Treatment related new adverse events after injection of the conjugate anti-GD2-IRDye800CW not to be expected from dinutuximab-alone infusion. AEs and treatment-emergent adverse events (TEAEs) will be summarized by body system and presented by severity and causal relationship to study drug. AE's will be coded using the Medical Dictionary for Regulatory Activities and listed.
Subjects experiencing serious AEs will be listed and summarized in tabular format.
Tumor to background ratio (TBR) of at least 2.0 ex vivo and sufficient for detection during surgery. TBR is established by dividing the mean fluorescence signal of the tumor by the mean fluorescence signal of the background. Background is determined by fluorescence intensity in healthy kidney, liver and surrounding background tissue.
The optimum dose is defined as the lowest dose of the administered immunoconjugate giving an adequate ex vivo TBR of >2.0.
We will compare the mean of the different groups with the Mann-Whitney U test. In case we find the same TBR's for different dose cohorts, we will choose the lowest dose with the highest TBR (minimal >2.0) for the recommended phase II dose.

### 10.2 Sample Size/Accrual Rate

Since this is a proof-of-principle study, first to be ever performed in children, we have chosen to test three different set doses in groups of a maximum of three-six patients. The sample size of this feasibility study will be 12-22 patients based on the Rolling-6 design for the safety part plus and expansion cohort of 10 patients treated at the recommended dose including the patients that enrolled in the safety part. This means that 12 patients will be included in case of adverse effects in dosage group 3 mg/m², (namely 6 patients in dosage group 1 mg/m², 2 patients in dosage group 3 mg/m² and an expansion cohort of 4 patients in dosage group 1 mg/m²). If there is no limitation by adverse effects, 22 patients will be included.

Currently, in the Princess Máxima Center, around 20 patients will undergo debulking surgery for neuroblastoma per year. We expect to achieve this number of potential candidates in 12-18 months. Therefore including interim safety evaluations, analysing the data and writing the study report the trial will be completed within 20-24 months.

### 10.3 Analysis of Secondary Endpoints

Changes in serum pharmacokinetics; blood samples will be taken at t=0 (just before infusion), t=24 h (after infusion), t=72 h (after infusion), t=96 h (immediately before surgery), t=120 h (24 h after surgery) and t=144 h (48 h after surgery). Dinutuximab levels and anti-drug-antibodies measured as described in section 6.7. Presence of anti-drug antibodies is a descriptive outcome measure. Dinutuximab level measurements will be combined and analyzed over time.
The sensitivity and specificity of our novel fluorescent agent to detect GD2-expressing, vital tumor cells. Pathologic status of removed fluorescent tissue and of removed non-fluorescent tissue will be compared to fluorescent status.
   Negative predictive value will be determined by the true negative lesions divided by the sum of the true negative lesions and the false negative lesions according to the pathologist.
   The positive predictive value will be determined by the true positive lesions divided by the sum of the true positive and the false positive lesions according to the pathologist.

### 11. Ethical Considerations

### 11.1 Patient Protection

The responsible investigator will ensure that this study is conducted in agreement with either the Declaration of Helsinki (Tokyo, Venice, Hong Kong, Somerset West and Edinburgh amendments) or the laws and regulations of the country, whichever provides the greatest protection of the patient.

The protocol has been written, and the study will be conducted according to the ICH Harmonised Tripartite Guideline Clinical for Good Practice (ref: http://www.ich.org/fileadmin/Public_Web_Site/ICH_Products/Guidelines/Efficacy/E6_R1/Step4/E6_R1_Guideline.pdf).

The protocol will be approved by the Local Ethics Committees.

### REFERENCES

1. Louis C U, Shohet J M. Neuroblastoma: molecular pathogenesis and therapy. Annu Rev Med. 2015; 66:49-63.
2. Shimada H, Ambros I M, Dehner L P, Hata J, Joshi V V, Roald B. Terminology and morphologic criteria of neuroblastic tumors: recommendations by the International Neuroblastoma Pathology Committee. Cancer. 1999; 86 (2): 349-63.
3. Jans M. W M, Van De Ven C., Van Baren R., Tytgat G., Zwaveling S. Analysis of Surgery for Neuroblastoma in The Netherlands. Pediatr Blood CancerSIOP 2016 Scientific Programme+Index.
4. Irwin M S, Park J R. Neuroblastoma: paradigm for precision medicine. Pediatr Clin North Am. 2015; 62 (1): 225-56.
5. von Allmen D, Davidoff A M, London W B, Van Ryn C, Haas-Kogan D A, Kreissman S G, et al. Impact of Extent of Resection on Local Control and Survival in Patients From the COG A3973 Study With High-Risk Neuroblastoma. J Clin Oncol. 2017; 35 (2): 208-16.
6. Nagaya T, Nakamura Y A, Choyke P L, Kobayashi H. Fluorescence-Guided Surgery. Front Oncol. 2017; 7:314.
7. Harlaar N J, Koller M, de Jongh S J, van Leeuwen B L, Hemmer P H, Kruijff S, et al. Molecular fluorescence-guided surgery of peritoneal carcinomatosis of colorectal origin: a single-centre feasibility study. Lancet Gastroenterol Hepatol. 2016; 1 (4): 283-90.
8. Lamberts L E, Koch M, de Jong J S, Adams A L L, Glatz J, Kranendonk M E G, et al. Tumor-Specific Uptake of Fluorescent Bevacizumab-IRDye800CW Microdosing in Patients with Primary Breast Cancer: A Phase I Feasibility Study. Clin Cancer Res. 2017; 23 (11): 2730-41.
9. Olson E S, Jiang T, Aguilera T A, Nguyen Q T, Ellies L G, Scadeng M, et al. Activatable cell penetrating peptides linked to nanoparticles as dual probes for in vivo fluorescence and M R imaging of proteases. Proc Natl Acad Sci USA. 2010; 107 (9): 4311-6.
10. Weissleder R, Tung C H, Mahmood U, Bogdanov A, Jr. In vivo imaging of tumors with protease-activated near-infrared fluorescent probes. Nat Biotechnol. 1999; 17 (4): 375-8.
11. Aaboud M, Aad G, Abbott B, Abdallah J, Abdinov O, Abeloos B, et al. Electron efficiency measurements with the ATLAS detector using 2012 LHC proton-proton collision data. Eur Phys J C Part Fields. 2017; 77 (3): 195.
12. Cheresh D A, Harper J R, Schulz G, Reisfeld R A. Localization of the gangliosides GD2 and GD3 in adhesion plaques and on the surface of human melanoma cells. Proc Natl Acad Sci USA. 1984; 81 (18): 5767-71.
13. Svennerholm L, Bostrom K, Fredman P, Jungbjer B, Lekman A, Mansson J E, et al. Gangliosides and allied glycosphingolipids in human peripheral nerve and spinal cord. Biochim Biophys Acta. 1994; 1214 (2): 115-23.
14. Yu A L, Gilman A L, Ozkaynak M F, London W B, Kreissman S G, Chen H X, et al. Anti-GD2 antibody with GM-CSF, interleukin-2, and isotretinoin for neuroblastoma. N Engl J Med. 2010; 363 (14): 1324-34.
15. Ter Weele E J, Terwisscha van Scheltinga A G, Linssen M D, Nagengast W B, Lindner I, Jorritsma-Smit A, et al. Development, preclinical safety, formulation, and stability of clinical grade bevacizumab-800CW, a new near infrared fluorescent imaging agent for first in human use. Eur J Pharm Biopharm. 2016; 104:226-34.
16. Gao R W, Teraphongphom N, de Boer E, van den Berg N S, Divi V, Kaplan M J, et al. Safety of panitumumab-IRDye800CW and cetuximab-IRDye800CW for fluorescence-guided surgical navigation in head and neck cancers. Theranostics. 2018; 8 (9): 2488-95.
17. Ozkaynak M F, Sondel P M, Krailo M D, Gan J, Javorsky B, Reisfeld R A, et al. Phase I study of chimeric human/murine anti-ganglioside G (D2) monoclonal antibody (ch14.18) with granulocyte-macrophage colony-stimulating factor in children with neuroblastoma immediately after hematopoietic stem-cell transplantation: a Children's Cancer Group Study. J Clin Oncol. 2000; 18 (24): 4077-85.
18. Gilman A L, Ozkaynak M F, Matthay K K, Krailo M, Yu A L, Gan J, et al. Phase I study of ch14.18 with granulocyte-macrophage colony-stimulating factor and interleukin-2 in children with neuroblastoma after autologous bone marrow transplantation or stem-cell rescue: a report from the Children's Oncology Group. J Clin Oncol. 2009; 27 (1): 85-91.
19. Reagan-Shaw S, Nihal M, Ahmad N. Dose translation from animal to human studies revisited. FASEB J. 2008; 22 (3): 659-61.
20. Yu A L, Uttenreuther-Fischer M M, Huang C S, Tsui C C, Gillies S D, Reisfeld R A, et al. Phase I trial of a human-mouse chimeric anti-disialoganglioside monoclonal antibody ch14.18 in patients with refractory neuroblastoma and osteosarcoma. J Clin Oncol. 1998; 16 (6): 2169-80.
21. Lu G, van den Berg N S, Martin B A, Nishio N, Hart Z P, van Keulen S, et al. Tumour-specific fluorescence-guided surgery for pancreatic cancer using panitumumab-IRDye800CW: a phase 1 single-centre, open-label, single-arm, dose-escalation study. Lancet Gastroenterol Hepatol. 2020.
22. Navid F, Sondel P M, Barfield R, Shulkin B L, Kaufman R A, Allay J A, et al. Phase I trial of a novel anti-GD2 monoclonal antibody, Hu14.18K322A, designed to decrease toxicity in children with refractory or recurrent neuroblastoma. J Clin Oncol. 2014; 32 (14): 1445-52.
23. Wellens L M, Deken M M, Sier C M, Johnson H R, Ortiz, F J, Bhairosigh S S, et al. Anti-GD2-IRDye800CW as a target for fluorescence-guided surgery in neuroblastoma. Nat Sci Rep. 2020; 10:17667
24. Tummers W S, Warram J M, Tiperneni K E, Fengeler J, Jacobs P, Lalitha S. Regulatory aspect of optical methods and exogenous targets for cancer detection. Cancer Res. 2017; 77 (9): 2197-2206.
25. Committee for Medicinal Products for Human Use (CHMP). Assessment report, Dinutuximab beta Apeiron, International non-proprietary name: dinutuximab beta. March 2017. Procedure No. EMEA/H/C/003918/0000
26. Summary of Product Characteristics. Qarziba 4.5 mg/mL concentrate for solution for infusion
27. Tummers W S, Warram J M, Van den Berg N S, Miller S E, Swijnenburg R J, Vahrmeijer A L et al. Theranostics. 2018; 8 (19): 5336-5347
28. Karin A van Schie, Gerrit-Jan Wolbink, Theo Rispens. Cross-reactive and pre-existing antibodies to therapeutic antibodies—Effects on treatment and immunogenicity. MAbs. 2015; 7 (4): 662-71.
29. Krishna M, Nadler S G. Immunogenicity to Biotherapeutics—The Role of Anti-drug Immune Complexes. Front Immunol. 2016 Feb. 2; 7:21.
30. El Amrani M, Göbel C, Egas A C, Nierkens S, Hack C E, Huitema A D R, van Maarseveen E M. Quantification of neutralizing anti-drug antibodies and their neutralizing capacity using competitive displacement and tandem mass spectrometry: Infliximab as proof of principle. J Transl Autoimmun. 2019 May 28; 1:100004.
31. Yu A L, Gilman A L, Ozkaynak M F, et al. Long-Term Follow-up of a Phase III Study of ch14.18 (Dinutuximab)+Cytokine Immunotherapy in Children with High-Risk Neuroblastoma: COG Study ANBL0032. Clin Cancer Res. 2021 Apr. 15; 27 (8): 2179-2189
32. Keyel M E, Reynolds C P. Spotlight on dinutuximab in the treatment of high-risk neuroblastoma: development and place in therapy. Biologics. 2018 Dec. 21; 13:1-12.
33. Vincent F B, Morand E F, Murphy K, Mackay F, Mariette X, Marcelli C. Antidrug antibodies (ADAb) to tumour necrosis factor (TNF)-specific neutralising agents in chronic inflammatory diseases: a real issue, a clinical perspective. Ann Rheum Dis. 2013 February; 72 (2): 165-78.
34. El Amrani M, Szanto C L, Hack C E, Huitema A D R, Nierkens S, van Maarseveen E M. Quantification of total dinutuximab concentrations in neuroblastoma patients with liquid chromatography tandem mass spectrometry. Anal Bioanal Chem. 2018; 410 (23): 5849-5858.
35. Siebert N, Eger C, Seidel D, Jüttner M, Zumpe M, Wegner D, et al. Pharmacokinetics and pharmacodynamics of ch14.18/CHO in relapsed/refractory high-risk neuroblastoma patients treated by long-term infusion in combination with IL-2. MAbs. 2016; 8 (3): 604-16.
36. Lamberts L E, Koch M, de Jong J S, Adams A L L, Glatz J, Kranendonk M E G et al. Tumor-Specific Uptake of Fluorescent Bevacizumab-IRDye800CW Microdosing in Patients with Primary Breast Cancer: A Phase I Feasibility Study. Clin Cancer Res. 2017; 23 (11): 2730-2741.
37. Miller S E, Tummers W S, Teraphongphom N, van den Berg N S, Hasan A, Ertsey R D, et al. First-in-human intraoperative near-infrared fluorescence imaging of glioblastoma using cetuximab-IRDye800. J Neurooncol. 2018; 139 (1): 135-143.

1. A GD2-specific fluorescent agent for use in a method of detecting a GD2 positive tumour in a human patient;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
wherein detecting the GD2 positive tumour comprises exposing the GD2 positive tumour to NIR light and detecting the fluorescence emission from the GD2 positive tumour.

2. The GD2-specific fluorescent agent for use of claim 1, wherein the GD2-specific fluorescent agent is administered in a dose of 1, 3, 10, 15 or 20 mg/m² body surface area.

3. The GD2-specific fluorescent agent for use of claim 1 or 2, wherein the GD2-specific fluorescent agent is administered by intravenous infusion, optionally over a period of 24 hours.

4. The GD2-specific fluorescent agent for use of any preceding claim, wherein administering the GD2-specific fluorescent agent is commenced 4 days prior to detecting the GD2 positive tumour.

5. The GD2-specific fluorescent agent for use of any preceding claim, wherein the ex vivo tumor to background ratio (TBR) of the fluorescence signal is at least 2.0.

6. The GD2-specific fluorescent agent for use of any preceding claim, wherein the sensitivity of the GD2-specific fluorescent agent for the detection of GD2-expressing tumor cells, typically GD2-expressing vital tumor cells, is at least 0.1, at least 0.2, at least 0.3, at least 0.4, at least 0.5, at least 0.6, at least 0.65, at least 0.7, at least 0.75, at least 0.8, at least 0.85, at least 0.9, or at least 0.95.

7. The GD2-specific fluorescent agent for use of any preceding claim, wherein the specificity of the GD2-specific fluorescent agent for the detection of GD2-expressing tumor cells, typically GD2-expressing vital tumor cells, is at least 0.5, at least 0.6, at least 0.65, at least 0.7, at least 0.75, at least 0.8, at least 0.85, at least 0.9, at least 0.95, at least 0.96, or at least 0.97, at least 0.98, or at least 0.99.

8. The GD2-specific fluorescent agent for use of any preceding claim, wherein the positive predictive value of the GD2-specific fluorescent agent for the detection of GD2-expressing tumor cells, typically GD2-expressing vital tumor cells, is at least 0.5, at least 0.6, at least 0.65, at least 0.7, at least 0.75, at least 0.8, at least 0.85, at least 0.9, at least 0.95, at least 0.96, or at least 0.97, at least 0.98, or at least 0.99.

9. The GD2-specific fluorescent agent for use of any preceding claim, wherein the ch14.18 antibody is dinutuximab beta.

10. The GD2-specific fluorescent agent for use of any one of claims 1 to 8, wherein the ch14.18 antibody is dinutuximab.

11. The GD2-specific fluorescent agent for use of any preceding claim, wherein the ch14.18 antibody is conjugated to the IRDye800CW by an amide bond.

12. The GD2-specific fluorescent agent for use of any preceding claim, wherein the patient has neuroblastoma, such as high risk neuroblastoma, intermediate risk neuroblastoma or low risk neuroblastoma; or wherein the patient has melanoma, a bone or soft-tissue sarcoma, small-cell lung cancer, renal cell carcinoma, a brain tumor, Ewing sarcoma, osteosarcoma, breast cancer, desmoplastic small round cell tumor, or retinoblastoma.

13. The GD2-specific fluorescent agent for use of claim 12, wherein the patient has newly-diagnosed, relapsed or refractory neuroblastoma.

14. The GD2-specific fluorescent agent for use of any preceding claim, wherein detecting the neuroblastoma comprises imaging the GD2 positive tumour.

15. A GD2-specific fluorescent agent for use in a method of diagnosing an area of resectable GD2 positive tumour in a human patient;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
wherein diagnosing an area of resectable GD2 positive tumour comprises exposing the GD2 positive tumour to NIR light and detecting the fluorescence emission from the GD2 positive tumour.

16. The GD2-specific fluorescent agent for use of claim 15, having any one of the additional features of claims 1-14.

17. A GD2-specific fluorescent agent for use in a method of fluorescence guided surgery to resect a GD2 positive tumour;
wherein the GD2-specific fluorescent agent comprises a conjugate of a near-infrared fluorescent dye IRDye800CW and a ch14.18 antibody; and
wherein the method comprises intravenously administering the GD2-specific fluorescent agent to the patient in a dose of from 1 to 20 mg/m² body surface area commencing between 1 day and 7 days prior to detecting of the GD2 positive tumour; and
detecting the GD2 positive tumour by exposing the GD2 positive tumour to NIR light and detecting the fluorescence emission from the GD2 positive tumour; and
surgical resection of the GD2 positive tumour detected in the patient.

18. The GD2-specific fluorescent agent for use of claim 17, wherein fluorescence guided surgery comprises surgical resection of all surgically resectable GD2 positive tumours detected in the patient.

19. The GD2-specific fluorescent agent for use of claim 18, wherein the method further comprises estimation of extent of tumor resection.

20. The GD2-specific fluorescent agent for use of claim 19, wherein if the resection extent is at least 90%, the patient is not treated with boost radiation therapy, whereas if the resection extent is less than 90%, the patient is treated with boost radiation therapy.

21. The GD2-specific fluorescent agent for use of any of claims 17 to 19, wherein the fluorescence guided surgery increases the proportion of patients for whom a resection extent of at least 90% can be achieved, compared to the proportion of patients having the same prognostic classification and receiving standard surgical resection, within the same overall treatment plan.

22. The GD2-specific fluorescent agent for use of any of claims 17 to 21, wherein the fluorescence guided surgery improves one or more clinical parameters of a cohort of patients compared to a cohort of patients having the same prognostic classification and receiving standard surgical resection, within the same overall treatment plan, optionally wherein the one of more clinical parameters are selected from overall response rate, complete response rate, partial response rate, primary tumour volume reduction, Curie score, event free survival and overall survival.

23. The GD2-specific fluorescent agent for use of any of claims 17 to 22, wherein the fluorescence guided surgery reduces the risk of surgical complications of a cohort of patients compared to a cohort of patients having the same prognostic classification and receiving standard surgical resection.

24. A method of imaging a GD2 positive tumour, the method comprising exposing the GD2 positive tumour to NIR light and detecting the fluorescence emission from the GD2 positive tumour,
wherein the patient has previously been administered a GD2-specific fluorescent agent in a dose of from 1 to 20 mg/m² body surface area commencing between 1 day and 7 days prior to imaging of the GD2 positive tumour; and wherein the GD2-specific fluorescent agent comprises a conjugate of a near-infrared fluorescent dye IRDye800CW and a ch14.18 antibody.

25. The method of claim 22 having any one of the additional features of claims 1 to 24.


SEQUENCE LISTING (4 sequences)

SEQ ID NO: 1 - DNA, 723 bp, synthetic construct
  misc_feature 1..723: note = artificial antibody chain
  source 1..723: mol_type = other DNA
atggaagccccagcgcagcttctcttcctcctgctactctggctcccagataccactggagaaatagtgatgacgcagtctccagccaccctgtctgtgtctccaggggaaagagccaccctctcctgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 1389 bp, synthetic construct
  misc_feature 1..1389: note = artificial antibody chain
  source 1..1389: mol_type = other DNA
atgggatggacctggatctttattttaatcctgtcggtaactacaggtgtccactctgaggtccaactgctgcagtctggacctgagctggagaagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 3 - protein, 240 aa, synthetic construct
  REGION 1..240: note = artificial antibody chain
MEAPAQLLFLLLLWLPDTTGEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 462 aa, synthetic construct
  REGION 1..462: note = artificial antibody chain
MGWTWIFILILSVTTGVHSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK